(19) United States
(12) Patent Application Publication
(10) Pub. No.: US 2026/0191553 A1
(43) Pub. Date: Jul. 9, 2026

(54) BALLOON, BALLOON CATHETER, ABLATION CATHETER, AND TUBULAR ULTRASONIC FOCUSING DEVICE AND METHOD

(71) Applicant: SHANGHAI HANTONG MEDICAL TECHNOLOGY CO., LTD., Shanghai (CN)

(72) Inventors: Wanjin Zhao, Shanghai (CN); Yong Wu, Shanghai (CN); Gang Peng, Shanghai (CN); Yi Fang, Shanghai (CN); Bo Zhang, Shanghai (CN)

(21) Appl. No.: 19/553,479

(22) Filed: Mar. 2, 2026

Related U.S. Application Data
(63) Continuation of application No. PCT/CN2025/104025, filed on Jun. 26, 2025.

(30) Foreign Application Priority Data
Sep. 11, 2024 (CN) 202411267299.7
May 30, 2025 (CN) 202510722105.6

(51) Int. Cl. A61B 17/32; A61B 17/00

(52) U.S. Cl. CPC A61B 17/320068; A61B 2017/00557; A61B 2017/320069

(57) ABSTRACT

The present disclosure provides a balloon, a balloon catheter, an ablation catheter, a tubular ultrasonic focusing device, and a method, and relates to the field of medical devices. The balloon in an expanded state includes: a first extension segment, a lens segment, and a second extension segment, wherein the lens segment is arranged between the first extension segment and the second extension segment in an axial direction, and the lens segment includes a converging structure prepared from an energy-permeable material, wherein the converging structure is configured to converge energy in the lens segment to a target convergence region. The balloon catheter includes a balloon, an energy generator, a first axial fixation member, and a second axial fixation member, wherein the energy generator is arranged in a hollow channel of the lens segment, and the energy generator has a length in an axial direction corresponding to that of the lens segment.

### CROSS-REFERENCE TO RELATED APPLICATION

The present disclosure is a Continuation Application of PCT Application No. PCT/CN2025/104025 filed on Jun. 26, 2025, which claims priority to Chinese patent application 202411267299.7, filed on Sep. 11, 2024, entitled “TUBULAR ULTRASONIC FOCUSING DEVICE AND METHOD”, and Chinese patent application 202510722105.6, filed on May 30, 2025, entitled “BALLOON, BALLOON CATHETER, AND ABLATION CATHETER”, the entire contents of which are incorporated herein by reference.

### TECHNICAL FIELD

The present disclosure relates to the fields of medical devices and ultrasonic ablation, and specifically to a balloon, a balloon catheter, an ablation catheter, a tubular ultrasonic focusing/weak-focusing device, and a method.

### BACKGROUND ART

Ablation is a medical technique that destroys or removes body tissues by physical or chemical means. Common ablations include: radiofrequency ablation, which uses high-frequency current to heat and destroy the target tissue; laser ablation, which uses laser energy to destroy the diseased tissue; cryoablation, which freezes and kills the diseased tissue by using extremely low temperatures (usually with liquid nitrogen or other cooling agents); and chemical ablation, which dissolves or destroys the diseased tissue by injecting chemical agents.

Energy is converged on the target tissue to damage the specific nerve fibers or nerve ganglia within the target tissue, thereby interrupting transmission and conduction of pathological nerve signals. This approach is used to treat diseases caused by excessive sympathetic nerve activity, abnormal pain transmission, or disorders of the autonomic nervous system (such as hypertension, arrhythmia, and chronic pain). Common neuro-ablation surgeries for the nervous system include: pulmonary artery denervation (PADN), renal denervation (RDN), and endovascular denervation (EDN). Besides these, there are various other neuro-ablation techniques, mainly targeting overactive or pathological neural pathways, and used to treat diseases such as refractory hypertension, arrhythmia, and pain syndromes.

When performing the above neuro-ablation surgeries, it is usually necessary to rely on the ablation catheter to perform the interventional surgery. The common ablation catheter is provided with an ablation energy release element on the catheter bracket. The ablation of different target positions is achieved through the design of the arrangement of the ablation energy release element.

In November 2023, the Paradise RDN system developed by ReCor Company of the US based on ultrasonic ablation technique, and the Symplicity Spyral RDN system developed by Medtronic of the US based on radiofrequency ablation technology, became the first two products to be approved by the US FDA for the treatment of primary hypertension.

In the Symplicity Spyral radiofrequency RDN system, the energy device consists of four annular metal electrodes, which are sequentially placed on a rollable memory metal guidewire. Regarding the ablation mechanism, the electrodes contact the vessel wall and apply a 448 kHz radiofrequency current to the vessel wall, and the current flows through the regional tissue and heats it up. Regarding the ablation depth, each electrode contacts the adjacent area less than 4 mm in size, which can achieve an ablation temperature exceeding 600. Regarding the ablation range: the ablation points are not on the same axial plane and only cover one quadrant in the projection direction. Regarding the vessel protection, there is no dedicated cooling mechanism, and it relies only on the cooling effect of the arterial blood.

In May 2024, the National Medical Products Administration of China approved the Medtronic Symplicity Spyral RDN system for use in treating resistant hypertension in the Chinese market, making it the first RDN product to be approved in the Chinese market.

In the Paradise ultrasonic RDN system, regarding the energy device: a tubular piezoelectric ceramic tube is placed inside a balloon that can accommodate the cooling liquid. Regarding the ablation mechanism: the balloon wall contacts the blood vessel; the tubular transducer emits 10 MHz ultrasonic energy radially; and the mechanical wave vibration causes the tissue to heat up. Regarding the ablation depth: the annular axis is symmetric; the axial length is 6 mm; and the radial outer diameter can reach 8 mm. Regarding the ablation range: it is similar to a “swimming ring” in a shape of 360-degree annular shape. Regarding the vascular protection: the balloon accommodates the coolant, thereby ensuring that the blood and the vascular wall within the <1 mm thickness region remains below 42° C. The traditional tubular transducer used in the product has an uneven radial distribution of acoustic field intensity, which approximately decays with the negative one exponent of the radius (r-1). This may result in excessive ablation in proximal areas within the target region, but ablation in distal areas is insufficient. Meanwhile, a part of the acoustic field can penetrate into more distant non-target areas, which results in a certain risk of tissue damage in the regions.

The existing ablation devices and methods indicate that the production process of the ablation energy release elements is quite challenging, and the development cycle is longer. Moreover, the energy convergence effect needs to be further enhanced.

### SUMMARY

An objective of embodiments of the present disclosure is to provide a balloon, a balloon catheter, and an ablation catheter. The balloon, balloon catheter, and ablation catheter achieve precise focusing on the target ablation area through a lens segment, which provides accurate targeting of nerves in the target ablation area, and also minimizes damage to surrounding tissues, thereby improving the effect of the ablation surgery.

In a first aspect, the embodiments of the present disclosure provide a balloon, and the balloon in an expanded state includes: a first extension segment, a lens segment, and a second extension segment, wherein the lens segment is arranged between the first extension segment and the second extension segment in an axial direction, and the lens segment includes a converging structure prepared from an energy-permeable material, wherein the converging structure is configured to converge energy from a hollow channel of the lens segment to a target convergence region.

In the above implementation, the balloon provided by the embodiments of the present disclosure includes the first extension segment, the lens segment, and the second extension segment, wherein the lens segment includes the converging structure, and the converging structure can converge the energy from the hollow channel of the lens segment to the target convergence region. Through the design of the converging structure, it is possible to realize the convergence of energy at different depths, i.e., to realize the adjustment of the focal point. The balloon structure provided by the embodiments of the present disclosure can be used in the catheter of interventional surgery. When used in conjunction with an energy generator, it can achieve precise ablation of the target ablation site. This approach not only achieves accurate energy focusing, but also improves the temperature rise speed at the ablation sites, shortens the duration of the ablation surgery, and improves the surgical experience of the patient.

Optionally, in the embodiments of the present disclosure, the converging structure includes a plurality of first thickness segments and a plurality of second thickness segments; radial dimensions of the first thickness segments and the second thickness segments are different; and the plurality of first thickness segments and the plurality of second thickness segments are arranged alternately in the axial direction.

In the above implementation, the converging structure of the balloon provided by the embodiments of the present disclosure includes the first thickness segments and the second thickness segments with different sizes in the radial direction. The first thickness segments and the second thickness segments are arranged alternately in the axial direction, which forms a binary thickness distribution of the balloon. The balloon provided by the embodiments of the present disclosure realizes the regulation of the energy distribution from the hollow channel of the balloon by providing the lens segment with the acoustic characteristics of a Bessel or a Fresnel lens. For the Bessel balloon, the first thickness segments and the second thickness segments have the same length in the axial direction. By adjusting the number of the first thickness segments and the second thickness segments, the control of the energy distribution from the hollow channel of the balloon can be achieved.

Optionally, in the embodiments of the present disclosure, the lens segment includes a middle length segment; and the middle length segment is arranged in an axial middle part of the lens segment, and the converging structure is arranged on two sides of the middle length segment.

In the above implementation, the balloon provided by the embodiments of the present disclosure includes a Fresnel balloon and a quasi-Bessel balloon. For the Fresnel balloon, the axial length can be designed based on the Fresnel zone plate formula and the position where energy convergence needs to be controlled. The quasi-Bessel lens is a simplified version of the Bessel lens structure. It achieves precise control of the position of the energy convergence by flexibly adjusting the length of the middle part. It can be seen that the balloon provided by the embodiments of the present disclosure can be endowed with acoustic structural characteristics of the Bessel lens, Fresnel lens, and quasi-Bessel lens. The flexible adjustment of its structural parameters can control the energy to converge to the target area. When applied to the catheter configuration in the ablation surgery, in combination with the use of the energy generator, it helps achieve precise ablation of the target ablation position.

Optionally, in the embodiments of the present disclosure, the balloon is a non-compliant balloon.

In the above implementation, during the ablation surgery, the non-compliant balloon can ensure that the ablation energy is evenly delivered to the target tissue through the tightly fitting to the balloon wall, which avoids uneven energy distribution or tissue damage due to excessive balloon expansion. In addition, its high burst pressure can safely withstand the high-pressure environment required for the surgery, and at the same time stably support the integrated electrode or transducer to accurately locate the ablation target, thereby enhancing the safety and effectiveness of the ablation surgery.

In a second aspect, the embodiments of the present disclosure provide a balloon catheter. The balloon catheter includes a balloon, an energy generator, a first axial fixation member, and a second axial fixation member, wherein the balloon in an expanded state includes: a first extension segment, a lens segment, and a second extension segment, wherein the lens segment is arranged between the first extension segment and the second extension segment in an axial direction; the energy generator is arranged in a hollow channel of the lens segment, and the energy generator has a length in an axial direction corresponding to that of the lens segment; the first axial fixation member is arranged in a hollow channel of the first extension segment and is fixedly connected to a first end of the energy generator; and the second axial fixation member is arranged in a hollow channel of the second extension segment and is fixedly connected to a second end of the energy generator, wherein a converging structure of the lens segment is configured to converge energy generated by the energy generator to a target convergence region.

In the above implementation, the embodiments of the present disclosure provide a balloon catheter, and the energy generator is fixed to the hollow channel of the lens segment of the balloon. The balloon includes the first extension segment, the lens segment, and the second extension segment, wherein the lens segment includes the converging structure, and the converging structure can converge the energy from the hollow channel of the lens segment to the target convergence region. Through the design of the converging structure, it is possible to realize the convergence of energy at different depths, i.e., to realize the adjustment of the focal point. The balloon catheter provided by the embodiments of the present disclosure can be used in the catheter of interventional surgery, which can achieve precise ablation of the target ablation site. This approach not only achieves accurate energy focusing, but also improves the temperature rise speed at the ablation sites, shortens the duration of the ablation surgery, and improves the surgical experience of the patient.

Optionally, in the embodiments of the present disclosure, the converging structure includes first thickness segments with a base thickness d1 and second thickness segment with a thickness d2, wherein the thickness d2 of the second thickness segments is obtained by adding a phase difference thickness d to the base thickness d1, and the phase difference thickness d is determined based on a phase difference between adjacent wave groups generated by the energy generator.

In the above implementation, the converging structure of the balloon in the balloon catheter provided by the embodiments of the present disclosure adopts a binary distribution thickness design, which can endow the lens segment with the acoustic characteristics of the Bessel or Fresnel lens. By adjusting the number of the first thickness segments and the second thickness segments, it is possible to achieve the regulation of the energy distribution (such as the energy emitted by the ultrasonic transducer) from the hollow channel of the balloon. By applying the balloon catheter provided by the embodiments of the present disclosure to the denervation ablation surgery, it is possible to precisely focus the energy on the target ablation area, thereby effectively reducing the duration of the ablation surgery, and enhancing the surgical comfort degree of the patient.

Optionally, in the embodiments of the present disclosure, lengths of the first thickness segments and the second thickness segments in the axial direction are approximate or equal to the phase difference thickness d.

In the above implementation, in the Bessel balloon catheter and the quasi-Bessel balloon catheter provided by the embodiments of the present disclosure, for each first thickness segment and the second thickness segment, the lengths thereof in the axial direction are set to be approximated or equal to the phase difference thickness d, so as to realize the precise notification of the linear phase delay and generate a non-diffracting Bessel beam.

Optionally, in the embodiments of the present disclosure, focusing characteristics presented by the converging structure are consistent with focusing characteristics of the quasi-Bessel lens; and the middle length segment of the lens segment is determined based on a position of the target convergence region.

In the above implementation, in the quasi-Bessel balloon catheter provided by the embodiments of the present disclosure, the focusing characteristics presented by the converging structure are consistent with focusing characteristics of the quasi-Bessel lens. The embodiments of the present disclosure take RDN surgery as an example to adjust the first thickness segment and the second thickness segment at the middle part of the Bessel balloon catheter. Through simulation, the quasi-Bessel balloon catheter provided by the embodiments of the present disclosure can achieve energy convergence at the target ablation area required for the RDN surgery. The structure is simple and highly manufacturable, which provides a strong and reliable support for the ablation surgery.

Optionally, in the embodiments of the present disclosure, the focusing characteristics presented by the converging structure are consistent with focusing characteristics of the—Bessel lens; and the number of the first thickness segments and the second thickness segments are n and n+1 respectively, wherein n is determined based on the position of the target convergence region.

In the above implementation, regarding the Bessel balloon catheter provided by the embodiments of the present disclosure, the converging structure is covered on the entire lens segment of the balloon. In the converging structure, the first thickness segments and the second thickness segments are alternately distributed, and the lengths of the first thickness segments and the second thickness segments in the axial direction are consistent. By adjusting the designed number of the first thickness segments and the second thickness segments, the energy distribution from the hollow channel of the balloon can be adjusted.

Optionally, in the embodiments of the present disclosure, the converging structure includes n first thickness segments and n+1 second thickness segments, wherein when a distance between a center point at the position of the target convergence region in a radial direction and an outer surface of the lens segment is in a range of [5.7 mm, 6.3 mm], n is set to 11.

In the above implementation, the balloon catheter provided by the embodiments of the present disclosure includes the Bessel balloon catheter, and the focusing characteristics presented by the converging structure are consistent with focusing characteristics of the Bessel lens. The embodiments of the present disclosure take RDN surgery as an example to design and adjust parameters. Through the simulation, the Bessel balloon catheter provided by the embodiments of the present disclosure can achieve energy convergence at the target ablation area required for the RDN surgery.

Optionally, in the embodiments of the present disclosure, the focusing characteristics presented by the converging structure are consistent with focusing characteristics of the Fresnel lens; and lengths of the first thickness segments, the second thickness segment, and a middle length segment in the axial direction are determined based on the position of the target convergence region according to the Fresnel zone plate formula.

In the above implementation, in the Fresnel balloon catheter provided by the embodiments of the present disclosure, the focusing characteristics presented by the converging structure are consistent with focusing characteristics of the Fresnel lens. The embodiments of the present disclosure take RDN surgery as an example to calculate the lengths of the first thickness segment and the second thickness segment at the middle part of the Fresnel balloon catheter. Through the simulation, the Fresnel balloon catheter provided by the embodiments of the present disclosure can achieve energy convergence at the target ablation area required for the RDN surgery. The structure is simple and highly manufacturable, which can significantly shorten the duration of the ablation surgery and provide reliable support for the ablation surgery.

Optionally, in the embodiments of the present disclosure, the position of the target convergence region is determined based on a target physiological position to be ablated.

Optionally, in the embodiments of the present disclosure, the target physiological position includes sympathetic nerve positions or parasympathetic nerve positions of a renal artery, duodenum, celiac artery, abdominal aorta, common hepatic artery, and hepatic innominate artery.

In the above implementation, the position of the target convergence region of the balloon catheter provided by the embodiments of the present disclosure is determined based on the target physiological position to be ablated, and the convergence position of the energy is determined based on the physiological position, which realizes precise ablation, thereby significantly enhancing the safety and effectiveness of the treatment. It can not only damage the target tissue, but also protect the surrounding healthy structures to the greatest extent. The balloon catheter provided by the embodiments of the present disclosure can significantly improve the surgical accuracy, efficiency, and safety of ablation surgeries for complex diseases, such as metabolic syndrome and refractory visceral pain, thereby promoting the development of minimally invasive surgeries towards higher precision.

Thirdly, the embodiments of the present disclosure provide an ablation catheter. The ablation catheter includes an energy generator, a first axial fixation member, a second axial fixation member, and a balloon having a first extension segment, a lens segment, and a second extension segment in its expanded state, wherein the lens segment is arranged between the first extension segment and the second extension segment; radial dimensions of the first extension segment and the second extension segment increase in an axial direction towards the lens section, and they are connected to the lens section; the energy generator is arranged in a hollow channel of the lens segment; the first axial fixation member is arranged in a hollow channel of the first extension segment and is fixedly connected to a first end of the energy generator; the second axial fixation member is arranged in a hollow channel of the second extension segment and is fixedly connected to a second end of the energy generator; and lengths of energy generator and the lens segment are equal in the axial direction, wherein a converging structure of the lens segment is configured to converge energy generated by the energy generator to a target convergence region.

In a fourth aspect, the present disclosure provides a tubular ultrasonic focusing/weak-focusing device including a balloon, wherein a transducer is arranged in the balloon; the transducer emits an acoustic beam for ablation; an acoustic beam adjustment structure is distributed along an axial direction of the balloon; and the acoustic beam passes through the balloon and the acoustic beam adjustment structure to form an adjustable focusing/weak-focusing region on an outer side of the balloon.

Optionally, the acoustic beam adjustment structure is formed by the lens segment according to the first aspect or the embodiments of the present disclosure, so as to modulate the acoustic beam passing therethrough by the converging structure prepared by an energy-permeable material in the lens segment, thereby forming a modulated convergence region or an adjustable focusing/weak-focusing region.

Optionally, the acoustic beam adjustment structure includes at least one or more acoustic unit structures, and the at least one or more acoustic unit structures are located on a balloon wall of the balloon.

Optionally, the acoustic beam adjustment structure includes at least one or more acoustic unit structures, and the at least one or more acoustic unit structures are located on an outer wall of the transducer.

Optionally, the acoustic beam adjustment structure includes at least one or more acoustic unit structures, and the at least one or more acoustic unit structures are located in a region between the transducer and the balloon.

Optionally, at least multiple groups of acoustic unit structures are symmetrically arranged along the axial direction of the balloon, and the at least multiple groups of acoustic unit structures are located at two ends of the balloon.

Optionally, the acoustic unit structure includes a first medium and a second medium, wherein the first medium and the second medium are arranged at intervals.

Optionally, the first medium and the second medium are similar in size.

Optionally, the first medium is in an annular shape and arranged axially along an outer wall of the balloon.

Optionally, the first medium and the balloon are made of a same material.

In a fifth aspect, the present disclosure provides a tubular ultrasonic focusing/weak-focusing method which uses the above tubular ultrasonic focusing/weak-focusing device to ablate the treatment target area.

In the fourth and fifth aspects of the present disclosure, by adjusting the ultrasonic frequency, the distance between the acoustic structure and the incident surface of the treatment area, and by adjusting the focusing/weak focusing region formed by the acoustic beam adjustment structure, the ablation depth and accuracy can be adjusted, and the synchronous temperature rise within the target area can be realized. Moreover, the tubular ultrasonic focusing/weak-focusing device and the tubular ultrasonic focusing/weak-focusing method provided by the present disclosure have advantages of uniform and consistent ablation effect, and a distinct distal boundary. It can achieve precise ablation for the renal artery branches and the main trunk, which ensures thorough ablation and minimizes damage to the surrounding normal tissues as much as possible. It meets the clinical requirement of “ablation as complete as possible”, thereby improving the effectiveness and safety of the treatment.

Other features and advantages of the present disclosure will be illustrated in the subsequent specification. The objects and other advantages of the present disclosure are realized and obtained by the structure particularly indicated in the written specification and drawings. In order to make the above purposes, features, and advantages of the present disclosure more obvious and easy to understand, the following is a detailed description of embodiments in conjunction with the drawings.

### BRIEF DESCRIPTION OF DRAWINGS

In order to more clearly illustrate the technical solutions in the embodiments of the present disclosure, the drawings to be used in the embodiments of the present disclosure will be briefly introduced below. It should be understood that the following drawings only show certain embodiments of the present disclosure, and therefore should not be regarded as a limitation of the scope. For a person of ordinary skill in the art, other relevant drawings can be obtained according to these drawings without inventive efforts.

FIG. 1 shows a structure schematic diagram of a traditional balloon;

FIG. 2 shows a structure schematic diagram of a Bessel balloon provided by the embodiments of the present disclosure;

FIG. 3 shows a structure schematic diagram of a Fresnel balloon provided by the embodiments of the present disclosure;

FIG. 4 shows a structure schematic diagram of a quasi-Bessel balloon provided by the embodiments of the present disclosure;

FIG. 5 shows a structure schematic diagram of a traditional balloon catheter;

FIG. 6 shows a diagram of acoustic simulation results of a traditional balloon catheter provided by embodiments of the present disclosure;

FIG. 7 shows a structure schematic diagram of a Bessel balloon catheter provided by the embodiments of the present disclosure;

FIG. 8 shows a structure schematic diagram of a Fresnel balloon catheter provided by the embodiments of the present disclosure;

FIG. 9 shows a structure schematic diagram of a quasi-Bessel balloon catheter provided by embodiments of the present disclosure;

FIG. 10 shows a diagram of acoustic simulation results of a Bessel balloon catheter provided by the embodiments of the present disclosure;

FIG. 11 shows a diagram of acoustic simulation results of a quasi-Bessel balloon catheter provided by the embodiments of the present disclosure;

FIG. 12 shows a diagram of acoustic simulation results of a Fresnel balloon catheter provided by the embodiments of the present disclosure;

FIG. 13 shows a diagram of bio-thermal simulation results of a traditional balloon catheter provided by the embodiments of the present disclosure;

FIG. 14 shows a diagram of bio-thermal simulation results of a Bessel balloon catheter provided by the embodiments of the present disclosure;

FIG. 15 shows a diagram of bio-thermal simulation results of a quasi-Bessel balloon catheter provided by the embodiments of the present disclosure;

FIG. 16 shows a diagram of bio-thermal simulation results of a Fresnel balloon catheter provided by the embodiments of the present disclosure;

FIG. 17 shows a diagram of temperature variation curves at a 6 mm radial position provided by the embodiments of the present disclosure;

FIG. 18 shows a temperature distribution schematic diagram at an axial center position of a transducer after 14 seconds provided by the embodiments of the present disclosure;

FIG. 19 shows a comparison diagram of a radial acoustic field distribution of a transducer provided by the embodiments of the present disclosure;

FIG. 20 shows a diagram of sound pressure increase at the 6 mm position according to the embodiments of the present disclosure.

FIG. 21 is a structure schematic diagram of a tubular ultrasonic weak-focusing device provided by the embodiments of the present disclosure;

FIG. 22 is a structure schematic diagram of an acoustic beam adjustment structure in a tubular ultrasonic weak-focusing device provided by the embodiments of the present disclosure;

FIG. 23 is a schematic diagram of a treatment state of an acoustic beam adjustment structure in a tubular ultrasonic weak-focusing device provided by the embodiments of the present disclosure;

FIG. 24 is a structure schematic diagram of a balloon in a tubular ultrasonic weak-focusing device provided by the embodiments of the present disclosure;

FIG. 25 is a schematic diagram of a comparison of acoustic fields in a tubular ultrasonic weak-focusing device provided by the embodiments of the present disclosure;

FIG. 26 is a schematic diagram of a comparison of temperature fields in a tubular ultrasonic weak-focusing device provided by the embodiments of the present disclosure;

FIG. 27 is a schematic diagram of a comparison of temperature rise in a tubular ultrasonic weak-focusing device provided by the embodiments of the present disclosure;

FIG. 28 is a schematic diagram of a comparison of ablation depths in a tubular ultrasonic weak-focusing device provided by the embodiments of the present disclosure; and

FIG. 29 is a schematic diagram of temperature rise at different radial points in a tubular ultrasonic weak-focusing device provided by the embodiments of the present disclosure.

Reference numbers: 1, balloon; 101, free propagation zone; 102, first coherence zone; 103, second coherence zone; 2, transducer; 3, inner tube; 4, first acoustic unit structure; 41, first medium; 42, second medium; 5, second acoustic unit structure. axial direction-Y; radial direction-R; balloon-100; first extension segment-110; lens segment-120; converging structure-121; first thickness segment-1211; second thickness segment-1212; middle length segment-122; second extension segment-130; balloon catheter-1000; energy generator-200; first axial fixation member-300; second axial fixation member-400.

### DETAILED DESCRIPTION OF EMBODIMENTS

In order to make the purpose, technical solutions and advantages of the examples of the present disclosure clearer, the technical solutions in the examples of the present disclosure will be described clearly and completely below in conjunction with drawings in the embodiments of the present disclosure. It is clear that the embodiments described are only some of embodiments of the present disclosure, and not all of the embodiments. The components of embodiments of the present disclosure generally described and shown in the drawings herein can be arranged and designed in various different configurations.

Therefore, the following detailed description of some of the embodiments of the present disclosure arranged in the drawings is not intended to limit the scope of the embodiments of the present disclosure for which protection is claimed, but only represents selected ones in the embodiments of the present disclosure. Based on some of the embodiments in present disclosure, all other examples obtained by a person of ordinary skill in the art without inventive efforts, all fall within the scope of protection of the embodiments of the present disclosure.

It should be noted that similar symbols and letters denote similar items in the following drawings, so that once an item is defined in a drawing, no further definition or explanation of it is required in the subsequent drawings.

In the description in the embodiments of the present disclosure, it should be noted that orientations or positional relationships indicated by terms, such as “center”, “up”, “down”, “left”, “right”, “vertical”, “horizontal”, “inside”, and “outside”, etc., are the orientations or positional relationships based on the drawings, or the orientation or positional relationship that the product of the embodiments of the present disclosure is customarily placed in use, which are only to facilitate the description of the embodiments of the present disclosure and simplify the description, and are not to indicate or imply that the device or element referred to must have a particular orientation, or be constructed and operated with a particular orientation, and therefore cannot to be understood as limitations of the embodiments of the present disclosure. Additionally, the terms “first”, “second”, and “third”, etc., are used only to distinguish descriptions, and are not to be understood as indicating or implying a relative importance.

Additionally, the terms “horizontal”, or “vertical”, etc., do not mean that the component must be absolutely horizontal or overhanging, but can be slightly inclined, e.g., “horizontal” only refers that it is more horizontal than “vertical” and does not mean that the structure must be absolutely horizontal, but can be slightly inclined.

In the description of the embodiments of the present disclosure, it should also be noted that unless other expressly specifications and limitations, the terms “arrange”, “mount”, “connect”, and “link” are to be understood in a broad sense, e.g. it can be a fixed connection, a detachable connection, or an integral connection; it can be a mechanical connection or an electrical connection; and it can be a direct connection, an indirect connection through an intermediate medium, or a communication inside two components. For those of ordinary skill in the art, the specific meaning of the above terms in the embodiments of the present disclosure can be understood in specific cases.

Ablation is a medical technique that destroys or removes body tissues by physical or chemical means. Energy is converged on the target tissue based on physical means to damage the specific nerve fibers or nerve ganglia within the target tissue, thereby interrupting transmission and conduction of pathological nerve signals. This approach is used to treat diseases caused by excessive sympathetic nerve activity, abnormal pain transmission, or disorders of the autonomic nervous system (such as hypertension, arrhythmia, and chronic pain). Common neuro-ablation surgeries for the nervous system include: pulmonary artery denervation (PADN), renal denervation (RDN), and endovascular denervation (EDN). Besides these, there are various other neuro-ablation techniques, mainly targeting overactive or pathological neural pathways, and used to treat diseases such as refractory hypertension, arrhythmia, and pain syndromes.

Currently, the common ablation catheter is provided with an ablation energy release element on the catheter bracket. The ablation of different target positions is achieved through the design of the arrangement of the ablation energy release element.

The inventors found that in this process, the production process of the ablation energy release elements is quite challenging, and the development cycle is longer. Moreover, the energy convergence effect needs to be further enhanced.

Based on this, the present disclosure provides a balloon, a balloon catheter, and an ablation catheter. The balloon includes a first extension segment, a lens segment, and a second extension segment, wherein the lens segment includes a converging structure prepared from an energy-permeable material, and the converging structure is configured to converge energy from a hollow channel of the lens segment to a target convergence region. Further, the energy generator is arranged in the hollow channel of the lens segment to form the balloon catheter provided by the embodiments of the present disclosure. The balloon catheter provides a remarkable focusing effect, so as to rapidly reach the ablation temperature, thereby shortening the duration of the ablation surgery and enhancing the comfort of the patient.

The following takes the pulmonary artery denervation (PADN), renal denervation (RDN), and endovascular denervation (EDN) as examples to briefly illustrate that the catheter provided by the embodiments of the present disclosure can be applied to neuroablation surgeries via biological lumens.

Renal denervation (RDN) is a minimally invasive treatment method for effectively controlling the resistant hypertension. It reduces hyperactive sympathetic nerve activity by using radiofrequency ablation, ultrasound, or microwave technology to disrupt the sympathetic nerves surrounding the renal arteries.

Endovascular denervation (EDN) is a novel minimally invasive surgery that improves glycemic control in patients with type 2 diabetes by ablating the sympathetic nerves surrounding the renal artery, duodenum, celiac artery, abdominal aorta, common hepatic artery, and proper hepatic artery.

Pulmonary hypertension (PH) is a clinical and pathophysiological syndrome derived from structural or functional changes in the pulmonary vasculature resulting from multiple heterogeneous diseases (etiologies) and different pathogenesis mechanisms. These changes lead to increased pulmonary vascular resistance and elevated pulmonary arterial pressure, ultimately progressing to right heart failure and even death. The pathological reasons leading to elevated pulmonary arterial pressure in pulmonary hypertension primarily include pulmonary arterial hypertension, left heart disease, lung diseases, pulmonary artery obstruction, and unknown or multifactorial mechanisms. Pulmonary artery denervation (PADN) is a percutaneous pulmonary artery interventional treatment technique. It uses specific catheters to transfer radiofrequency energy to the sympathetic nerves on the adventitia of the pulmonary artery, causing the disappearance of nerve myelin sheaths and the fusion of axons, thereby inhibiting sympathetic nerve activity, increasing cardiac output, reducing pulmonary artery pressure, inhibiting pathological remodeling of the pulmonary artery, and improving the exercise endurance of patients and cardiac function.

In addition, the catheter provided by the embodiments of the present disclosure can be applied to ablation surgeries via biological lumens. It can damage specific nerve fibers or nerve ganglia of the target tissue to interrupt the transmission of pathological nerve signals, thereby treating diseases caused by excessive sympathetic nerve activity, abnormal pain transmission, or autonomic nerve dysfunction.

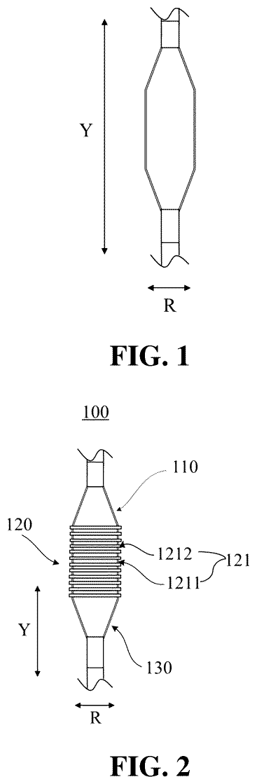

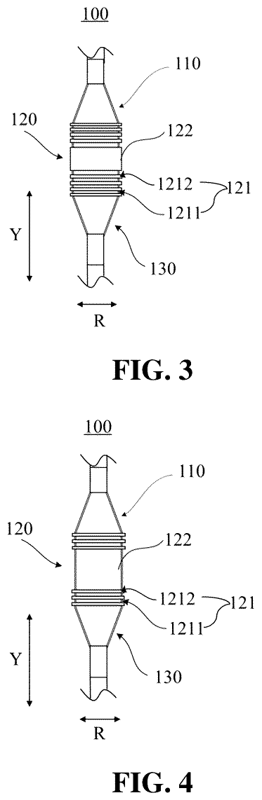

Referring to FIG. 1 to FIG. 4, FIG. 1 shows a structure schematic diagram of a traditional balloon; FIG. 2 shows a structure schematic diagram of a Bessel balloon provided by the embodiments of the present disclosure; FIG. 3 shows a structure schematic diagram of a Fresnel balloon provided by the embodiments of the present disclosure; and FIG. 4 shows a structure schematic diagram of a quasi-Bessel balloon provided by the embodiments of the present disclosure. It is noted that FIG. 1 to FIG. 4 all show the balloon in its expanded state.

A balloon 100 is a hollow device made of flexible material that can be inflated and deflated. It expands by the injection of liquid or gas and has wide medical applications. Common materials for balloon 100 include at least one of polyethylene (PE), polyethylene terephthalate (PET), polyamide (PA), and polyether block amide (Pebax).

In conjunction with FIG. 1, FIG. 1 shows a schematic diagram of a traditional balloon. The balloon in FIG. 1 is a conical balloon, which means that the radial dimensions at two ends are larger than the radial dimension in the middle. Commonly, there are cylindrical balloons and spherical balloons, etc., and the middle part of the balloon is usually the operation section. However, the balloon provided by the embodiment of the present disclosure adopts a lens-type design to its operation segment, which endows the balloon with the structural characteristics of a specialized lens to achieve control of energy. It should be understood that the shape of the balloon (such as conical, spherical, and cylindrical) shall not limit the scope of protection for the balloon provided by the embodiments of the present disclosure.

In conjunction with FIG. 2 to FIG. 4, the present disclosure provides a balloon 100. The balloon 100 in its expanded state includes: a first extension segment 110, a lens segment 120, and a second extension segment 130. It should be noted that the first extension segment 110 and the second extension segment 130 are the two ends of the balloon 100. In the application scenario of the medical device, the first extension segment 110 and the second extension segment 130 respectively correspond to the distal end (one end of the medical device close to the operator) and the proximal end (one end of the medical device away from the operator). The lens segment 120 is a portion with a lens configuration, and is generally arranged on an operation segment of the balloon 100. It should be noted that the lens segment 120 of the balloon 100 provided by embodiments of the present disclosure is a structure of the balloon 100 provided with a lens configuration, rather than a lens being arranged on the balloon 100.

As shown in FIG. 2 to FIG. 4, the lens segment 120 is arranged between the first extension segment 110 and the second extension segment 130 in an axial direction (Y). One end of the first extension segment 110 close to the lens segment 120 is connected to the lens segment 120, and one end of the second extension segment 130 close to the lens segment 120 is connected to the lens segment 120, so as to form the balloon 100.

The lens segment 120 includes a converging structure 121 prepared from an energy-permeable material, wherein the converging structure 121 is configured to converge energy from a hollow channel of the lens segment 120 to a target convergence region.

In the above implementation, The converging structure 121 of the lens segment 120 is made of an energy-permeable material. The preparation material can be selected from common preparation materials of the balloon 100, such as polyethylene (PE), polyethylene terephthalate (PET), polyamide (PA), and polyether block amide (Pebax).

As can be seen from FIG. 1 to FIG. 4, the balloon 100 provided by the embodiments of the present disclosure includes the first extension segment 110, the lens segment 120, and the second extension segment 130, wherein the lens segment 120 includes the converging structure 121, and the converging structure 121 can converge the energy from the hollow channel of the lens segment 120 to the target convergence region. Through the design of the converging structure 121, it is possible to realize the convergence of energy at different depths, i.e., to realize the adjustment of the focal point. The structure of the balloon 100 provided by the embodiments of the present disclosure can be used in the catheter of interventional surgery. When used in conjunction with an energy generator 200, it can achieve precise ablation of the target ablation site. This approach not only achieves accurate energy focusing, but also improves the temperature rise speed at the ablation sites, shortens the duration of the ablation surgery, and improves the surgical experience of the patient.

Continuing to refer to FIG. 2 to FIG. 4, in an optional embodiment of the embodiments of the present disclosure, the converging structure 121 includes a plurality of first thickness segments 1211 and a plurality of second thickness segments 1212.

The radial dimensions of the first thickness segments 1211 and the second thickness segments 1212 are different. As shown in FIG. 2 to FIG. 4, the plurality of first thickness segments 1211 and the plurality of second thickness segments 1212 are arranged alternately in the axial direction (Y).

In the above implementation, the first thickness segments 1211 and the second thickness segments 1212 have different dimensions in the radial direction (R), and the first thickness segments 1211 and the second thickness segments 1212 are alternatively arranged in the axial direction (Y), so that the balloon 100 provided by the embodiments of the present disclosure has a binary thickness periodic functional distribution in the axial direction (Y). For example, the arrangement pattern in the axial direction (Y) can be as follows: first thickness segment 1211 -second thickness segment 1212—first thickness segment 1211—second thickness segment 1212 . . . first thickness segment 1211—second thickness segment 1212—first thickness segment 1211; or second thickness segment 1212—first thickness segment 1211—second thickness segment 1212 . . . first thickness segment 1211—second thickness segment 1212.

Based on the above binary distribution thickness design, it can endow the lens segment 120 with the acoustic characteristics of the Bessel or Fresnel lens, thereby achieving the regulation of the energy distribution from the hollow channel of the balloon 100.

The Fresnel lens is a thin optical component that achieves the focusing or divergence of light through a concentric ring band structure. It discretizes the continuous curved surface of the traditional lens into stepped ring bands, which utilizes the principles of diffraction and refraction to reduce the thickness and to maintain the optical performance. Its characteristics are lightweight, but it has dispersion and efficiency losses.

Fresnel binary distribution is a diffraction optical design that discretely simulates the refraction effect of traditional lenses through the concentric ring band. The core of the design is to utilize the abrupt structural changes at the edge of the ring band to generate an optical path difference, which approximately achieves a secondary phase profile, thereby achieving the focusing function within a thin and planar structure, so as to effectively concentrate energy beams without the need for complex geometric designs.

The Bessel lens is a special optical component capable of generating non-diffracting Bessel beams. The Bessel beams are renowned for their non-diffraction characteristics, and can generate local sound waves to form standing wave patterns, which are also known as “frozen waves”. The Bessel lens constructs a conical wavefront that enables the beam to maintain a narrow diameter and long focal distance depth during propagation, thereby overcoming the diffraction spreading in conventional Gaussian beams.

The Bessel binary distribution generates an approximate conical phase through a discretized ring band structure, which is used to generate non-diffracting Bessel beams. This design modulates the wavefront by the annular diffraction, so as to form the beam with a long focal distance depth in the axial direction.

As shown in FIG. 2, FIG. 2 shows a structure schematic diagram of a Bessel balloon provided by the embodiments of the present disclosure. In FIG. 2, the converging structure 121 is covered on the lens segment 120 of the entire balloon 100. In the converging structure 121, the first thickness segments 1211 and the second thickness segments 1212 are alternately distributed, and the lengths of the first thickness segments 1211 and the second thickness segments 1212 in the axial direction (Y) are consistent. By adjusting the designed number of the first thickness segments 1211 and the second thickness segments 1212, the energy distribution from the hollow channel of the balloon 100 can be adjusted.

As can be seen that the converging structure 121 of the balloon 100 provided by the embodiments of the present disclosure includes the first thickness segments 1211 and the second thickness segments 1212 with different sizes in the radial direction (R), and the first thickness segments 1211 and the second thickness segments 1212 are arranged alternately in the axial direction (Y), which forms a binary thickness distribution of the balloon 100. The balloon 100 provided by the embodiments of the present disclosure realizes the regulation of the energy distribution from the hollow channel of the balloon 100 by providing the lens segment 120 with the acoustic characteristics of the Bessel or Fresnel lens. For the Bessel balloon 100, the first thickness segments 1211 and the second thickness segments 1212 have the same length in the axial direction (Y). By adjusting the number of the first thickness segments 1211 and the second thickness segments 1212, the control of the energy distribution from the hollow channel of the balloon 100 can be achieved.

Continuing to refer to FIG. 3 and FIG. 4, in an optional embodiment implemented by the present disclosure, the lens segment 120 further includes a middle length segment 122.

As shown in FIG. 3 and FIG. 4, the middle length segment 122 is arranged in an axial middle part of the lens segment 120, and the converging structure 121 is arranged on two sides of the middle length segment 122.

FIG. 3 shows a structure schematic diagram of a Fresnel balloon 100. In FIG. 3, the converging structure 121 is uniformly and symmetrically arranged on two sides of the middle length segment 122, i.e., the first thickness segments 1211 and the second thickness segments 1212 are alternately and symmetrically arranged on two sides of the middle length segment 122. In the Fresnel balloon 100 as shown in FIG. 3, the lengths of the first thickness segments 1211 and the second thickness segments 1212 in the axial direction (Y) are determined based on the position of the target convergence region that needs to control the energy convergence according to the Fresnel zone plate formula.

FIG. 4 shows a structure schematic diagram of a quasi-Bessel balloon 100. It should be noted that the quasi-Bessel lens is a specially designed optical component that can approximately provide key characteristics of the Bessel beam (such as non-diffraction, self-repairing, and long focal distance depth), and by simplifying the structure, it solves the problems of complexity and energy loss of the traditional Bessel beam generation method. The quasi-Bessel balloon 100 shown in FIG. 4 is a simplified version of the structure in FIG. 2. The first thickness segment 1211 and the second thickness segment 1212 in the focusing structure of the lens segment 120 in FIG. 2 are removed, thereby providing the above middle length segment 122. The lengths of the first thickness segments 1211 and second thickness segments 1212 on two sides of the middle length segment 122 are the same in the axial direction (Y). Through designing the number of the first thickness segments 1211 and the second thickness segments 1212 remaining on two sides, the modulation of the energy distribution from the hollow channel of the balloon 100 can be achieved.

As can be seen from FIG. 3 and FIG. 4, the balloon provided 100 by the embodiments of the present disclosure includes a Fresnel balloon 100 and a quasi-Bessel balloon 100. For the Fresnel balloon 100, the length in the axial direction (Y) can be designed based on the Fresnel zone plate formula and the position where energy convergence needs to be controlled. The quasi-Bessel lens is a simplified version of the Bessel lens structure. It achieves precise control of the position of the energy convergence by flexibly adjusting the length of the middle part. It can be seen that the balloon 100 provided by the embodiments of the present disclosure can be endowed with acoustic structural characteristics of the Bessel lens, Fresnel lens, and quasi-Bessel lens. The flexible adjustment of its structural parameters can control the energy to converge to the target area. When applied to the catheter configuration in the ablation surgery, in combination with the use of the energy generator, it helps achieve precise ablation of the target ablation position.

The balloon 100 provided by the embodiments of the present disclosure is a non-compliant balloon (NC). Non-compliant balloons are typically made of high-strength and low-tensile materials (such as PET). Under the high-pressure environment, its diameter hardly changes with the increase in pressure, so as to precisely maintain the preset size.

During the ablation surgery, the non-compliant balloon can ensure that the ablation energy is evenly delivered to the target tissue through the tightly fitting to the balloon wall, which avoids uneven energy distribution or tissue damage due to excessive balloon expansion. In addition, its high burst pressure can safely withstand the high-pressure environment required for the surgery, and at the same time stably support the integrated electrode or transducer to accurately locate the ablation target, thereby enhancing the safety and effectiveness of the ablation surgery.

The present disclosure further provides a balloon catheter 1000, and the balloon catheter 1000 includes the above balloon 100. Before introducing the specific content of the balloon catheter 1000 provided by the embodiments of the present disclosure, it needs to state in advance that in the embodiments of the present disclosure, simulation experiments on the balloon catheter 1000 were performed to verify the reliability of the balloon catheter 1000, including acoustic simulation and biological thermal simulation.

In order to perform the simulation experiment to verify the reliability of the balloon catheter 1000 provided by the embodiments of the present disclosure, a simulation model was first established. Modeling could be performed by using COMSOL Multiphysics (COMSOL Multiphysics, Burlington, MA, USA). The modeling process is not described in detail herein. However, to enable a person skilled in the art to reconstruct the structure of the balloon catheter 1000 provided by the embodiments of the present disclosure, the present disclosure provides the relevant parameters used in the modeling process (using the tubular ultrasonic transducer), which are shown in Table 1.

TABLE 1

| | |
|---|---|
| Parameter type | Numerical value |

| Ultrasonic transducer inner diameter | 1 | mm |
|---|---|---|
| Ultrasonic transducer outer diameter | 1.5 | mm |
| Axial length of the ultrasonic transducer | 6 | mm |
| Simulated arterial inner diameter | 4 | mm |
| Simulated arterial inner diameter | 6 | mm |
| Simulated tissue thickness | 12 | mm |
| Absorption of ultrasonic by water | default value | |
| Ultrasonic attenuation via arteries and tissues | 50.5 | NP/m² |
| Voltage | 30 | V |

It should be noted that all the simulation experiments provided by the embodiments of the present disclosure were performed under the same voltage/power excitation conditions.

For the ultrasonic transducer, the operating frequency of the ultrasonic transducer in the simulation experiments of the embodiments of the present disclosure was 8.5 MHz. Since the transducer operates independently of the balloon 100, changes in the design of the balloon 100 will not affect the performance of the transducer. Under the same excitation power, the sound field generated by different balloon 100 types remains consistent.

For the balloon 100 used in the simulation, the diameter was 4 mm. Clinically, due to the difference in vessel size, different sizes of the balloon 100 in a range of 4 millimeters to 8 millimeters can be required. As the radius of the balloon 100 changes, the distance between the balloon 100 and the transducer changes. However, as verified by simulation experiments, it was found that there was no significant change in the sound field distribution characteristics when the size of the balloon 100 was varied in the range of 4 millimeters to 8 millimeters.

In addition, the waves emitted by the transducer are similar to the cylindrical wavefront. Due to the minimal ultrasonic attenuation effect of the water circulating within the balloon 100, a divergence effect reducing the intensity may occur as the radius increases, but the effect on the shape of the sound field is negligible. It should be noted that the tubular piezoelectric ceramic transducer used in the embodiments of the present disclosure, in practical applications, the use of energy generators 200 of other shapes such as prismatic shape, is also within the protection scope of the embodiments of the present disclosure.

It should be specifically noted that during the bio-thermal simulation (in the subsequent FIG. 13 to FIG. 16) in the embodiments of the present disclosure, the effectiveness and safety thresholds of ultrasound ablation as defined by IEC 60601-2-62 (Medical electrical device-Part 2-62: special requirements for the basic safety and basic performance of high intensity ultrasonic ultrasonic (HITU) device were used. The effective ablation threshold was characterized by a thermal dose (T43) of 1800 seconds, and the safety threshold corresponded to a thermal dose (T43) of 60 seconds. Within the isothermal range of 54° C., the thermal dose of T43 exceeded 1800 seconds, meeting the criteria for effective ablation. Outside of the 48° C. isotherm, the thermal dose of T43 might be less than 60 seconds, remaining below the safety threshold and ensuring the safety of adjacent tissues. The embodiments of the present disclosure respectively simulated durations required for several types of balloon catheters 1000 provided by the embodiments of the present disclosure to reach 54° C. at a 6 mm ablation boundary (the duration required for the ablation surgery), in order to study the temperature rise condition; and the embodiments of the present disclosure simulated the distance between the 48° C. isothermal line and the vascular wall for the several types of balloon catheters 1000 provided by the embodiments within a given ablation duration, in order to study the impact on non-target areas.

The following is the specific content of the balloon catheter 1000 provided by the embodiments of the present disclosure.

The balloon catheter 1000 includes a balloon 100, an energy generator 200, a first axial fixation member 300, and a second axial fixation member 400, wherein the balloon 100 includes the balloon 100 provided in the first aspect of the embodiments of the present disclosure, and the balloon 100 in the expanded state can be referred to the foregoing description, including: a first extension segment 110, a lens segment 120, and a second extension segment 130. The lens segment 120 is arranged between the first extension segment 110 and the second extension segment 130 in an axial direction (Y).

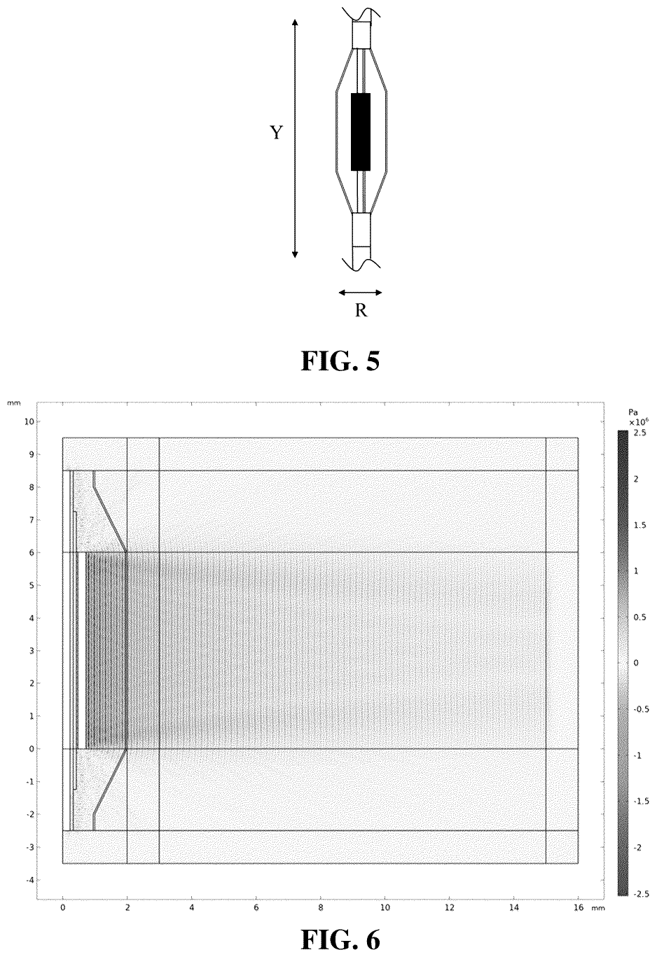

The structure of the balloon catheter 1000 provided by the embodiments of the present disclosure is described by taking a structure schematic diagram of a traditional balloon catheter as an example. Referring to FIG. 5, FIG. 5 shows a structure schematic diagram of a traditional balloon catheter 0. As shown in FIG. 5, the traditional balloon catheter 0 is provided with the energy generator 200 within a hollow channel in the middle of the balloon 100.

For the energy generator 200, in particular, the piezoelectric tubular transducer utilizes radially polarized piezoelectric ceramics to show an efficient capability of emitting ultrasonic power from the central axis to the exterior. This feature achieves 360° energy convergence, and is applied in medical applications of acoustic therapy within the cavity (such as renal denervation ablation, RDN), which can enhance the efficiency and effectiveness of the surgery.

In FIG. 5, the transducer at the middle of the balloon catheter 1000 is a tubular piezoelectric ceramic transducer. Referring to FIG. 6 and FIG. 13 in conjunction with FIG. 5, FIG. 6 shows a diagram of acoustic simulation results of a traditional balloon catheter provided by embodiments of the present disclosure, and FIG. 13 shows a diagram of bio-thermal simulation results of a traditional balloon catheter provided by the embodiments of the present disclosure. In FIG. 6, the horizontal axis represents the radial dimension (unit: mm); the left vertical axis represents the axial dimension (unit: mm); and the right vertical axis represents the energy intensity (unit: Pa). As can be seen from FIG. 6, the focusing effect of the traditional balloon catheter is poor, and the energy is in a divergent state. As shown in the bio-thermal simulation result shown in FIG. 13, it can be seen that the ablation duration required by the traditional balloon catheter is 18 seconds, and the distance between the 48° C. isotherm of the traditional balloon catheter and the vascular wall is 8.1 mm.

In the balloon catheter 1000 provided by the embodiments of the present disclosure, the energy generator 200 is arranged in a hollow channel of the lens segment 120, and the energy generator 200 has a length in an axial direction (Y) corresponding to that of the lens segment 120. It is to be noted that the corresponding length means that the length of the lens segment 120 can be determined according to the length of the energy generator 200. Optionally, the length of the lens segment 120 in the axial direction (Y) can be set to be the same as the length of the energy generator 200; or optionally, the length of the lens segment 120 in the axial direction (Y) can be set to be slightly smaller than the length of the energy generator 200.

The first axial fixation member 300 is arranged in a hollow channel of the first extension segment 110 and is fixedly connected to a first end of the energy generator 200; the second axial fixation member 400 is arranged in a hollow channel of the second extension segment 130 and is fixedly connected to a second end of the energy generator 200. The fixation method of the energy generator 200 can be referred to in FIG. 5. It is similar to the fixation method of the energy generator of the traditional balloon catheter. Two ends of the energy generator 200 are fixed by the first axial fixation member 300 and the second axial fixation member 400.

The converging structure 121 of the lens segment 120 is configured to converge energy generated by the energy generator 200 to a target convergence region.

Unlike the traditional balloon catheter, in the balloon catheter 1000 provided by the embodiments of the present disclosure, the energy generator 200 is fixed to the hollow channel of the lens segment 120 of the balloon 100. The balloon 100 includes the first extension segment 110, the lens segment 120, and the second extension segment 130, wherein the lens segment 120 includes the converging structure 121, and the converging structure 121 can converge the energy from the hollow channel of the lens segment 120 to the target convergence region. Through the design of the converging structure 121, it is possible to realize the convergence of energy at different depths, i.e., to realize the adjustment of the focal point. The balloon catheter 1000 provided by the embodiments of the present disclosure can be used in the catheter of interventional surgery, which can achieve precise ablation of the target ablation site. This approach not only achieves accurate energy focusing, but also improves the temperature rise speed at the ablation sites, shortens the duration of the ablation surgery, and improves the surgical experience of the patient.

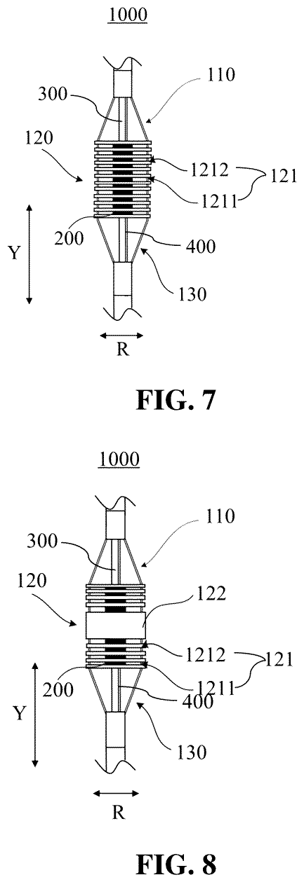

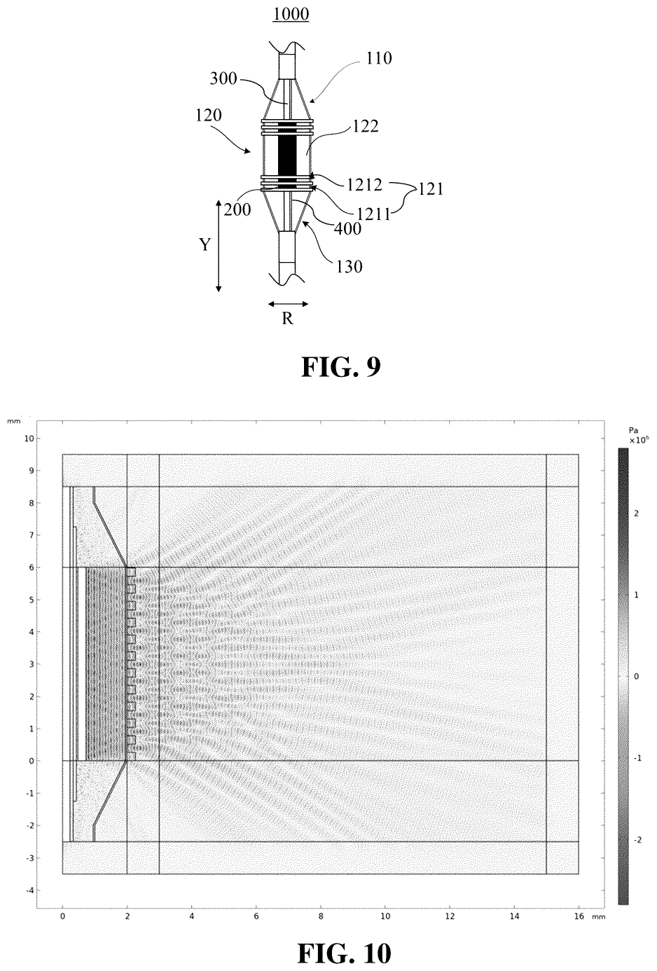

Referring to FIG. 7 to FIG. 9, FIG. 7 shows a structure schematic diagram of a Bessel balloon catheter provided by the embodiments of the present disclosure; FIG. 8 shows a structure schematic diagram of a Fresnel balloon catheter provided by the embodiments of the present disclosure; and FIG. 9 shows a structure schematic diagram of a quasi-Bessel balloon catheter provided by the embodiments of the present disclosure. In the balloon catheter 1000 provided by the embodiments of the present disclosure, the converging structure 121 of the balloon 100 includes the first thickness segments 1211 with the base thickness d1 and the second thickness segments 1212 with the thickness d2.

The thickness d2 of the second thickness segments 1212 is obtained by adding a phase difference thickness d to the base thickness d1, i.e., d2=d1+d; and the phase difference thickness d is determined based on a phase difference between adjacent wave groups generated by the energy generator 200.

The Bessel lens reconstructs the incident wavefront into a conical wavefront by precisely controlling the phase delay difference between adjacent ring wave groups, thereby generating the non-diffraction Bessel beam. Specifically, in the embodiments of the present disclosure, the radial thickness of the balloon 100 is set to different dimensions (the first thickness segments 1211 and the second thickness segments 1212), and specific phase differences are introduced to the light waves at different thickness positions, which causes the wavefront to form the concentric annular interference during propagation. Ultimately, the central bright spot (the target convergence region) of the Bessel beam is superimposed along the axis (perpendicular to the axial direction (Y)).

Exemplarily, taking the ultrasonic transducer as an example, the phase difference Δφ=π is defined. The phase difference thickness d can be obtained based on the following formula:

$$d = \frac{{c}_{w} {c}_{p}}{2 ( {c}_{p} - {c}_{w} ) f} ,$$

where c_w represents a speed of the ultrasound propagating in the tissue; c_p represents a speed of the ultrasound propagating in the balloon 100; and f represents an operating frequency. In addition, for traditional balloons, the base thickness d1 is typically 0.05 mm. It takes the balloon 100 with a diameter of 4 mm and targets a focal length of 6 mm (in denervation ablation surgeries, the nerve distribution is considered most concentrated at this position. Taking RDN surgery as an example, the target ablation area is within a radial range of 6 mm from the artery, which can effectively cover more than 95% of the renal artery nerve). It takes c_w=1500 m/s, c_p=2250 m/s, and f=8.5 MHz. Substituting them into the above formula, the calculated phase difference thickness d is 0.265 mm.

As can be seen from FIG. 7 to FIG. 9, the converging structure 121 of the balloon 100 in the balloon catheter 1000 provided by the embodiments of the present disclosure adopts the binary distribution thickness design, which can endow the lens segment 120 with the acoustic characteristics of the Bessel or Fresnel lens. By adjusting the number of the first thickness segments 1211 and the second thickness segments 1212, it is possible to achieve the regulation of the energy distribution (such as the energy emitted by the ultrasonic transducer) from the hollow channel of the balloon 100. By applying the balloon catheter 1000 provided by the embodiments of the present disclosure to the denervation ablation surgery, it is possible to precisely focus the energy on the target ablation area, thereby effectively reducing the duration of the ablation surgery, and enhancing the surgical comfort degree of the patient.

Referring to FIG. 7 and FIG. 9, FIG. 7 shows a Bessel balloon catheter 1000 and FIG. 9 shows a quasi-Bessel balloon catheter 1000, wherein the lengths of the first thickness segments 1211 and the second thickness segments 1212 in the axial direction (Y) are approximate or equal to the phase difference thickness d.

Exemplarily, in the above realization process, the calculated phase difference thickness d is 0.265 mm, and it can then be combined with the axial length of the ultrasonic transducer to design the length of each first thickness segment 1211 and second thickness segment 1212 in the axial direction (Y). For example, through calculation, the length of each first thickness segment 1211 and second thickness segment 1212 in the axial direction (Y) can be set to 0.26 mm.

In the Bessel balloon catheter 1000 and the quasi-Bessel balloon catheter 1000 provided by the embodiments of the present disclosure, for each first thickness segment 1211 and the second thickness segment 1212, the lengths thereof in the axial direction (Y) are set to be approximated or equal to the phase difference thickness d, so as to realize the precise notification of the linear phase delay and generate the non-diffracting Bessel beam.

Continuing to refer to FIG. 7, in the balloon catheter 1000 provided by the embodiments of the present disclosure, the focusing characteristics presented by the converging structure 121 of the Bessel balloon catheter 1000 are consistent with focusing characteristics of the Bessel lens.

The number of the first thickness segments 1211 and the second thickness segments 1212 are n and n+1 respectively, wherein n is determined based on the position of the target convergence region. As an example, in FIG. 7, the number of first thickness segments 1211 is n, and the number of second thickness segments 1212 is n+1. The corresponding arrangement pattern in the axial direction (Y) is as follows: second thickness segment 1212—first thickness segment 1211—second thickness segment 1212 . . . first thickness segment 1211—second thickness segment 1212. In some possible embodiments, the number of first thickness segments 1211 can be set to n+1, and the number of second thickness segments 1212 can be set to n. The corresponding arrangement pattern in the axial direction (Y) can be as follows: first thickness segment 1211—second thickness segment 1212—first thickness segment 1211—second thickness segment 1212 . . . first thickness segment 1211—second thickness segment 1212—first thickness segment 1211.

As can be seen that regarding the Bessel balloon catheter 1000 provided by the embodiments of the present disclosure, the converging structure 121 is covered on the entire lens segment 120 of the balloon 100. In the converging structure 121, the first thickness segments 1211 and the second thickness segments 1212 are alternately distributed, and the lengths of the first thickness segments 1211 and the second thickness segments 1212 in the axial direction (Y) are consistent. By adjusting the designed number of the first thickness segments 1211 and the second thickness segments 1212, the energy distribution from the hollow channel of the balloon 100 can be adjusted.

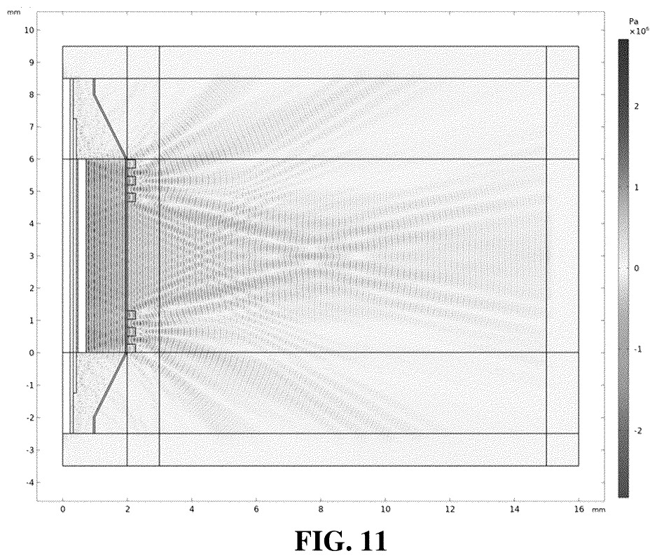

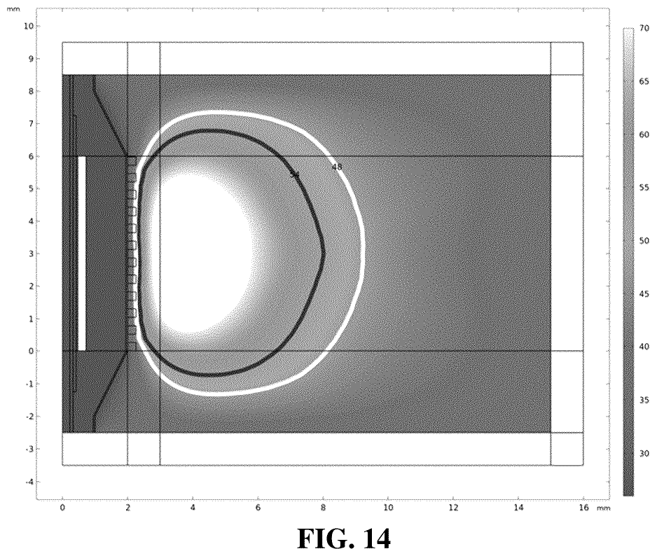

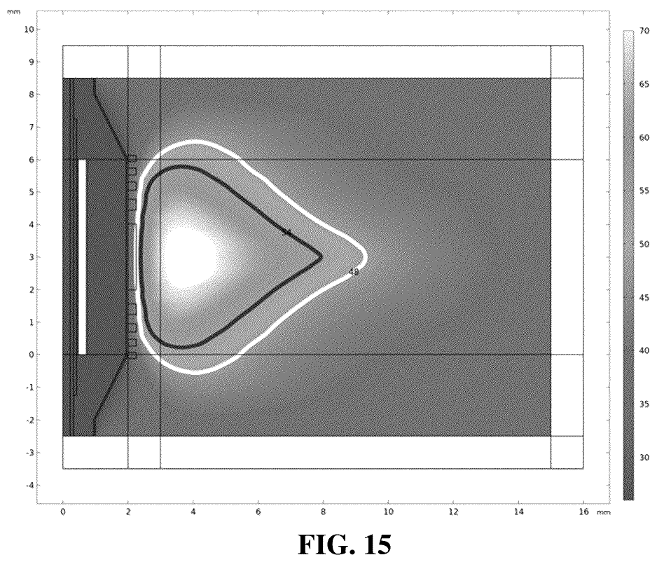

Referring to FIG. 10 and FIG. 14 based on FIG. 7, FIG. 10 shows a diagram of acoustic simulation results of the Bessel balloon catheter provided by the embodiments of the present disclosure; and FIG. 14 shows a diagram of bio-thermal simulation results of the Bessel balloon catheter provided by the embodiments of the present disclosure. In an optional embodiment of among the embodiments of the present disclosure, the converging structure 121 includes n first thickness segments 1211 and n+1 second thickness segments 1212.

When a distance between a center point at the position of the target convergence region in a radial direction (R) and an outer surface of the lens segment 120 is in a range of [5.7 mm, 6.3 mm], n is set to 11. Preferably, the distance between the center point at the position of the target convergence region in the radial direction (R) and the outer surface of the lens segment 120 at the base thickness is optimally controlled at 6 mm.

Taking the structure shown in FIG. 7 as an example, according to the requirements of RDN, the target ablation area is in a radial range of 6 mm from the artery, which effectively covers more than 95% of the renal artery nerves. For the Bessel balloon 100, the embodiments of the present disclosure use equal first thickness segments 1211 and second thickness segments 1212 of 0.26 millimeter (approximating the phase difference thickness d, the above phase difference thickness d is calculated to be 0.265), and it visually presents an “annular gap” structure. In FIG. 7, there are a total of 12 second thickness segments 1212 and 11 first thickness segments 1211 on the balloon 100, which can achieve the expected focal length of 6 mm.

In order to verify the reliability of the structure of FIG. 7, the embodiments of the present disclosure performed the acoustic simulation. The result of the acoustic simulation of the Bessel balloon catheter 1000 is shown in FIG. 10. In FIG. 10, the horizontal axis represents the radial dimension (unit: mm); the left vertical axis represents the axial dimension (unit: mm); and the right vertical axis represents the energy intensity (unit: Pa). As can be seen from FIG. 10, the Bessel lens balloon 100 exhibits a triangular convergence mode at a depth of 6 mm, which can concentrate the energy on the target ablation area required for RDN surgery.

Further, the bio-thermal simulation was performed, and the bio-thermal simulation result is shown in FIG. 14. As shown in FIG. 14, it can be seen that the ablation duration required by the Bessel balloon catheter 1000 is 25 seconds, and the distance between the 48° C. isotherm of the Bessel balloon catheter 1000 and the vascular wall is 7.1 mm. The impact on the non-target area is very small, so that the safe ablation surgery can be achieved.

As can be seen from FIG. 7, FIG. 10, and FIG. 14, the balloon catheter 1000 provided by the embodiments of the present disclosure includes the Bessel balloon catheter 1000, and the focusing characteristics presented by the converging structure 121 are consistent with focusing characteristics of the Bessel lens. The embodiments of the present disclosure take RDN surgery as an example to design and adjust parameters. Through the simulation, the Bessel balloon catheter 1000 provided by the embodiments of the present disclosure can achieve energy convergence at the target ablation area required for the RDN surgery.

Referring to FIG. 11 and FIG. 15 based on FIG. 9, FIG. 11 shows a diagram of acoustic simulation results of the quasi-Bessel balloon catheter provided by the embodiments of the present disclosure; and FIG. 15 shows a diagram of bio-thermal simulation results of the quasi-Bessel balloon catheter provided by the embodiments of the present disclosure. In the balloon catheter 1000 provided by the embodiments of the present disclosure, the focusing characteristics presented by the converging structure 121 of the quasi-Bessel balloon catheter 1000 are consistent with focusing characteristics of the quasi-Bessel lens.

FIG. 9 shows a structure schematic diagram of the quasi-Bessel balloon catheter 1000. It should be noted that the quasi-Bessel lens can approximately provide the key characteristics of the Bessel beam, and by simplifying the structure, it solves the problems of complexity and energy loss of the traditional Bessel beam generation method. As shown in FIG. 9, the quasi-Bessel balloon 100 is a simplified version of the Bessel balloon 100 structure shown in FIG. 7. The first thickness segment 1211 and the second thickness segment 1212 in the focusing structure of the lens segment 120 in FIG. 7 are removed, thereby providing the above middle length segment 122. The lengths of the first thickness segments 1211 and second thickness segments 1212 on two sides of the middle length segment 122 are the same in the axial direction (Y). Through designing the number of the first thickness segments 1211 and the second thickness segments 1212 remaining on two sides, the modulation of the energy distribution from the energy generator 200 in the hollow channel of the balloon 100 can be achieved.

In the above implementation, the middle length segment 122 of the lens segment 120 is determined based on a position of the target convergence region.

Still taking RDN surgery as an example, the target ablation area is in a radial range of 6 mm from the artery, which effectively covers more than 95% of the renal artery nerves. Taking the quasi-Bessel balloon 100 shown in FIG. 9 as an example, three rings on each side (the second thickness segments 1212) are retained, and the six central rings (the second thickness segments 1212) are removed. Thereby, the transducer is effectively divided into upper, middle, and lower parts, wherein the middle part corresponds to the peripheral tissue region, which is the main ablation target area. The ultrasonic waves emitted from this part of the transducer directly penetrate and interact with the target area. Meanwhile, the transducer segments located on the upper and lower parts adopt the Bessel acoustic structure, which helps to refract some of the energy to the middle target area. Therefore, the middle target area receives more energy than the original energy.

In order to verify the reliability of the balloon catheter 1000 shown in FIG. 9, the embodiments of the present disclosure provide a schematic diagram of the acoustic simulation result of the structure in FIG. 9. The result of the acoustic simulation of the quasi-Bessel balloon catheter 1000 is shown in FIG. 11. In FIG. 11, the horizontal axis represents the radial dimension (unit: mm); the left vertical axis represents the axial dimension (unit: mm); and the right vertical axis represents the energy intensity (unit: Pa). As can be seen in FIG. 11, the quasi-Bessel lens balloon 100 converges significantly at a depth of 6 mm, which can achieve the energy convergence at the target ablation area required for the RDN surgery.

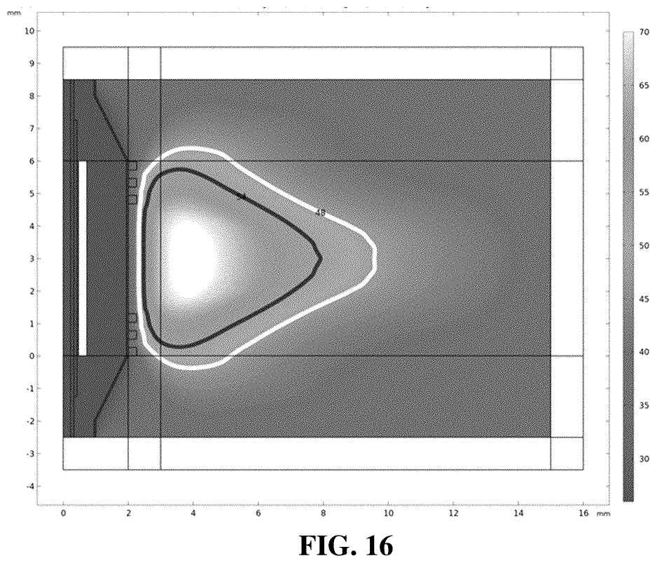

Further, the bio-thermal simulation was performed, and the bio-thermal simulation result is shown in FIG. 16. It can be seen from FIG. 16 that the ablation duration required by the quasi-Bessel balloon catheter 1000 is 14 seconds, which is about 22% less than the ablation duration of the traditional balloon catheter 0; and the distance between the 48° C. isotherm of the quasi-Bessel balloon catheter 1000 and the vascular wall is 7.8 mm. The impact on the non-target area is relatively small, so that the safe ablation surgery can be achieved.

As can be seen from FIG. 9, FIG. 11, and FIG. 16, in the quasi-Bessel balloon catheter 1000 provided by the embodiments of the present disclosure, the focusing characteristics presented by the converging structure 121 are consistent with focusing characteristics of the quasi-Bessel lens. The embodiments of the present disclosure take RDN surgery as an example to adjust the first thickness segment 1211 and the second thickness segment 1212 at the middle part of the Bessel balloon catheter 1000. Through simulation, the quasi-Bessel balloon catheter 1000 provided by the embodiments of the present disclosure can achieve energy convergence at the target ablation area required for the RDN surgery. The structure is simple and highly manufacturable, which provides a strong and reliable support for the ablation surgery.

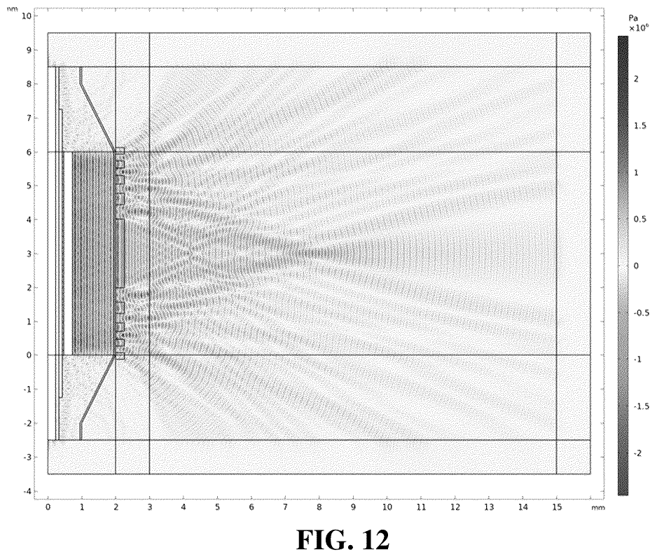

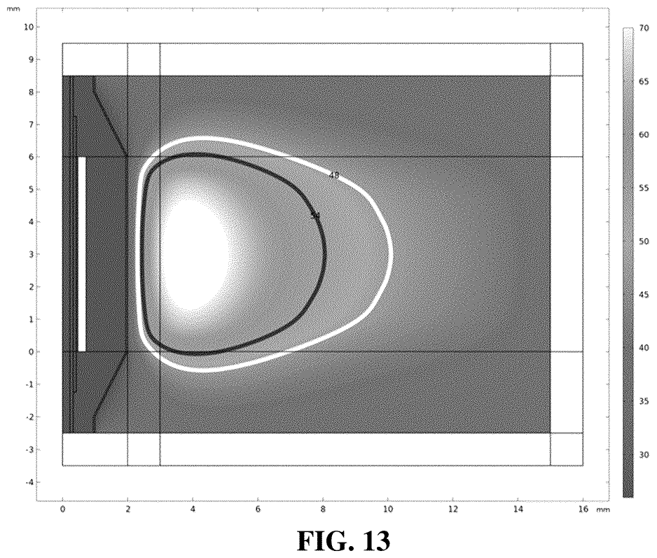

Referring to FIG. 12 and FIG. 15 based on FIG. 8, FIG. 12 shows a diagram of acoustic simulation results of the Fresnel balloon catheter provided by the embodiments of the present disclosure; and FIG. 15 shows a diagram of bio-thermal simulation results of the Fresnel balloon catheter provided by the embodiments of the present disclosure. In the balloon catheter 1000 provided by the embodiments of the present disclosure, the focusing characteristics presented by the converging structure 121 of the Fresnel balloon catheter 1000 are consistent with focusing characteristics of the Fresnel lens.

In the embodiment of the present disclosure, the lengths of the first thickness segments 1211, the second thickness segment 1212, and the middle length segment 122 in the axial direction (Y) are determined based on the position of the target convergence region according to the Fresnel zone plate formula.

In the embodiments of the present disclosure, for the Fresnel balloon catheter 1000, the length in the axial direction (Y) is determined based on the following formula:

$${r}_{n} = \sqrt{n λ {F}_{L} + {( \frac{n λ}{2} )}^{2}} , n = 1 , 2 , 3 … N ,$$

where F_L is a focal depth of 6 mm, and λ is the wavelength of ultrasonic waves in the tissue. Based on the length of the transducer (in Table 1), the maximum value of N is 7. Therefore, the path length from the “focal point” to the boundary of any ring (the second thickness segments 1212) is longer than the focal length F_L by an integer multiple of half the wavelength λ.

In order to verify the reliability of the balloon catheter 1000 shown in FIG. 8, the embodiments of the present disclosure provide a schematic diagram of the acoustic simulation result of the structure in FIG. 8. The result of the acoustic simulation of the Fresnel balloon catheter 1000 is shown in FIG. 12. In FIG. 12, the horizontal axis represents the radial dimension (unit: mm); the left vertical axis represents the axial dimension (unit: mm); and the right vertical axis represents the energy intensity (unit: Pa). As can be seen in FIG. 12, the Fresnel balloon 100 converges significantly at a depth of 6 mm, which can achieve the energy convergence at the target ablation area required for the RDN surgery.

Further, the bio-thermal simulation was performed, and the bio-thermal simulation result is shown in FIG. 15. It can be seen from FIG. 15 that the ablation duration required by the quasi-Bessel balloon catheter 1000 is 14 seconds, which is about 22% less than the ablation duration of the traditional balloon catheter 0; and the distance between the 48° C. isotherm of the Fresnel balloon catheter 1000 and the vascular wall is 7.3 mm. The impact on the non-target area is relatively small, so that the safe ablation surgery can be achieved.

As can be seen from FIG. 8, FIG. 12, and FIG. 15, in the Fresnel balloon catheter 1000 provided by the embodiments of the present disclosure, the focusing characteristics presented by the converging structure 121 are consistent with focusing characteristics of the Fresnel lens. The embodiments of the present disclosure take RDN surgery as an example to calculate the lengths of the first thickness segment 1211 and the second thickness segment 1212 at the middle part of the Fresnel balloon catheter 1000. Through simulation, it can be verified that the Fresnel balloon catheter 1000 provided by the embodiments of the present disclosure can achieve energy convergence at the target ablation area required for the RDN surgery. The structure is simple and highly manufacturable, which can significantly reduce the ablation surgery duration, so as to provide a strong and reliable support for the ablation surgery.

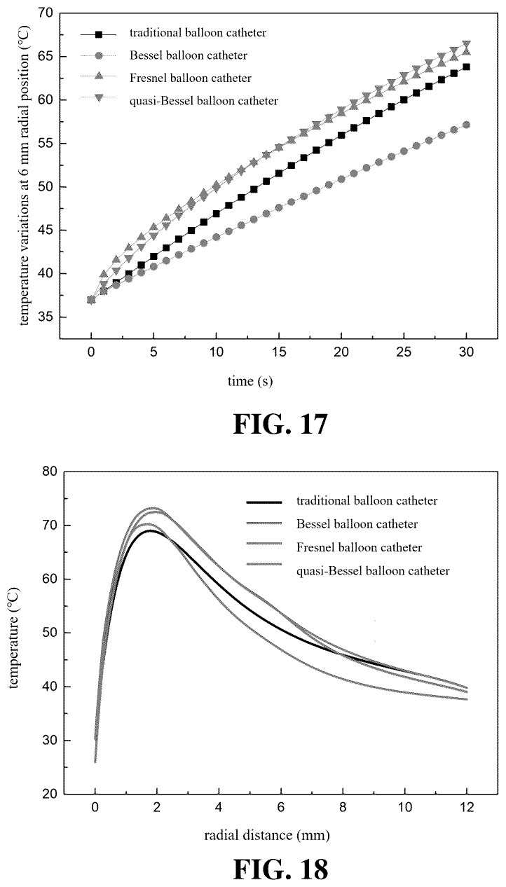

Referring to FIG. 17 based on FIG. 5 to FIG. 8, FIG. 17 shows a diagram of temperature variation curves at a 6 mm radial position provided by the embodiments of the present disclosure. In FIG. 17, the horizontal axis represents time (unit: s), and the vertical axis represents temperature variation at the 6 mm radial position (unit: ° C.). The line with black square markers indicates the temperature variation curve of the traditional balloon catheter 0; the line with red circular markers indicates the temperature variation curve of the Bessel balloon catheter 1000; the line with blue upright triangular markers indicates the temperature variation curve of the Fresnel balloon catheter 1000; and the line with pink purple inverted triangular markers indicates the temperature variation curve of the quasi-Bessel balloon catheter 1000. As can be seen in FIG. 17, the Fresnel balloon catheter 1000 and the quasi-Bessel balloon catheter 1000 have the same and highest slopes, which indicate the fastest rate of temperature increase. The temperatures of these two types of balloons 100 are 2-4° C. higher than those of traditional balloon catheters 0, which provides a significant advantage in reducing the ablation surgery duration.

Referring to FIG. 18 based on FIG. 5 to FIG. 8, FIG. 18 shows a temperature distribution schematic diagram at the axial center position of the transducer after 14 seconds provided by the embodiments of the present disclosure. In FIG. 18, the horizontal axis represents radial distance (unit: mm), and the vertical axis represents temperature (unit: ° C.). The line with black square markers indicates the temperature curve of the traditional balloon catheter 0; the line with red circular markers indicates the temperature curve of the Bessel balloon catheter 1000; the line with blue upright triangular markers indicates the temperature curve of the Fresnel balloon catheter 1000; and the line with pink purple inverted triangular markers indicates the temperature curve of the quasi-Bessel balloon catheter 1000. As can be seen in FIG. 18, the temperatures of the four balloons 100 are similar in the range of 1.5 mm. In the range of 1.5-2.5 mm, the Fresnel balloon catheter 1000 and the quasi-Bessel balloon catheter 1000 have the highest and similar temperatures, and the temperature of the Bessel balloon catheter 1000 is slightly lower but still higher than that of the traditional balloon. In the range of 2.5-6 mm, the temperatures of the Fresnel balloon catheter 1000 and the quasi-Bessel balloon catheter 1000 are similar, and they are about 3° C. higher than that of the traditional balloon. The temperature of the Bessel balloon catheter 1000 is slightly lower than that of the traditional balloon. Beyond 6 mm, the temperatures of the Fresnel balloon catheter 1000, the quasi-Bessel balloon catheter 1000, and the traditional balloon catheter 0 are similar.

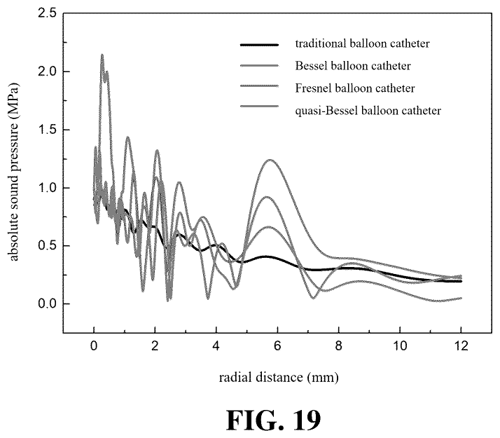

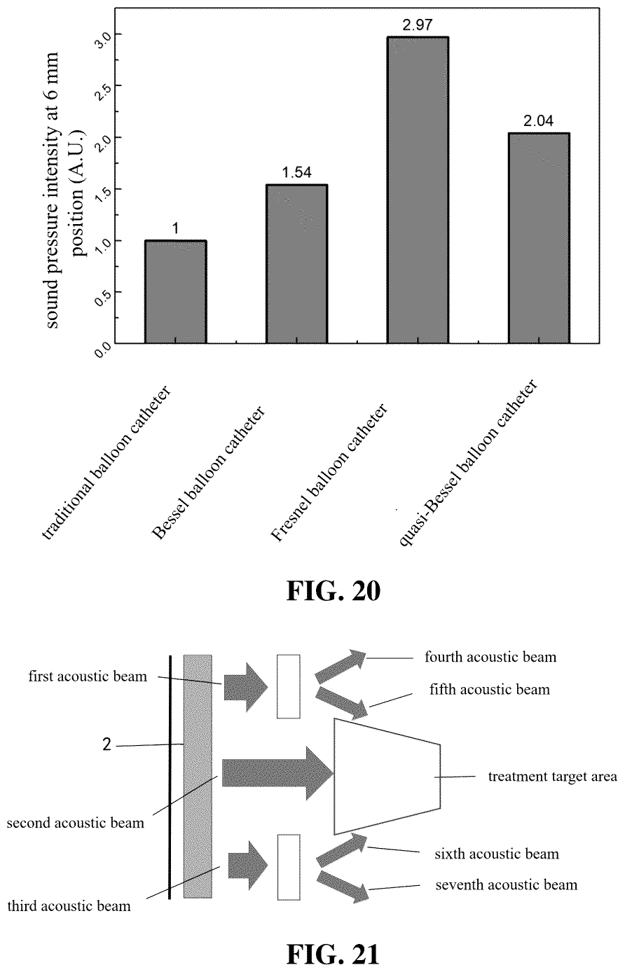

In conjunction with FIG. 19 and FIG. 20, FIG. 19 shows a comparison diagram of a radial acoustic field distribution of the transducer provided by the embodiments of the present disclosure; and FIG. 20 shows a diagram of sound pressure increase at the 6 mm position according to the embodiments of the present disclosure. In FIG. 19, the horizontal axis represents the radial distance (unit: mm), and the vertical axis represents the beam sound pressure (unit: MPa). The black line indicates the absolute sound pressure curve of the traditional balloon catheter 0; the red line indicates the absolute sound pressure curve of the Bessel balloon catheter 1000; the blue line indicates the absolute sound pressure curve of the Fresnel balloon catheter 1000; and the pink purple line indicates the absolute sound pressure curve of the quasi-Bessel balloon catheter 1000. In FIG. 20, the vertical axis represents the sound pressure intensity at 6 mm (units, A.U.) The red column indicates the sound pressure intensity of the traditional balloon catheter 0; the green column indicates the sound pressure intensity of the Bessel balloon catheter 1000; the dark blue column indicates the sound pressure intensity of the Fresnel balloon catheter 1000; and the light blue column indicates the sound pressure intensity of the quasi-Bessel balloon catheter 1000.

It is apparent from the figure that the balloon 100 provided with a lens shows a waveform peak at about 6 mm compared to the traditional balloon. As shown in FIG. 20, the sound pressure intensity at the 6 mm is compared with that of the traditional balloon after the normalization processing. Observation reveals that the Fresnel lens balloon 100 exhibits the highest sound intensity increase, approximately twice that of the traditional balloon. This is followed by the quasi-Bessel lens balloon 100 with an increase of 100%. The Bessel lens balloon 100 shows an increase of about 50%. Compared with the traditional balloon, the three lens balloons 100 all show significant sound intensity peaks at 6 mm.

As can be seen from the above simulation results, the Fresnel balloon catheter 1000 and the quasi-Bessel balloon catheter 1000 provided by the embodiments of the present disclosure have significantly better temperature rise effect compared to the traditional balloon catheter, and can reach the effective ablation temperature in a shorter duration. From a clinical point of view, this means that the patient pain tolerance duration is reduced and the overall treatment comfort is improved.

In an optional embodiment of among the embodiments of the present disclosure, the position of the target convergence region is determined based on a target physiological position to be ablated.

Taking RDN (renal artery denervation) as an example, when treating the resistant hypertension, the ablation energy needs to precisely target the sympathetic nerve fibers in the adventitia of the renal artery. The ablation catheter (such as radiofrequency or the ultrasonic balloon catheter 1000 provided by the embodiments of the present disclosure) will concentrate energy on a specific circumferential area of the vascular wall, so as to form a ring ablation band, which disrupts excessive active nerve signal transmission, and avoids damage to the inner membrane of the blood vessel or the adjacent renal parenchyma.

Optionally, the target physiological position in the embodiments of the present disclosure includes sympathetic nerve positions or parasympathetic nerve positions of a renal artery, duodenum, celiac artery, abdominal aorta, common hepatic artery, and hepatic innominate artery.

The position of the target convergence region of the balloon catheter 1000 provided by the embodiments of the present disclosure is determined based on the target physiological position to be ablated, and the convergence position of the energy is determined based on the physiological position, which realizes precise ablation, thereby significantly enhancing the safety and effectiveness of the treatment. It can not only damage the target tissue, but also protect the surrounding healthy structures to the greatest extent. The balloon catheter 1000 provided by the embodiments of the present disclosure can significantly improve the surgical accuracy, efficiency, and safety of ablation surgeries for complex diseases, such as metabolic syndrome and refractory visceral pain, thereby promoting the development of minimally invasive surgeries towards higher precision.

The embodiments of the present disclosure further provide an ablation catheter. The ablation catheter includes an energy generator, a first axial fixation member, a second axial fixation member, and a balloon having a first extension segment, a lens segment, and a second extension segment in its expanded state.

The lens segment is arranged between the first extension segment and the second extension segment; and radial dimensions of the first extension segment and the second extension segment increase in an axial direction towards the lens section, and they are connected to the lens section. As shown in the balloon structure in FIG. 7 to FIG. 9, the first extension segment and the second extension segment are conical, and the radial dimensions gradually increase towards the lens segment.

The energy generator is arranged in a hollow channel of the lens segment; the first axial fixation member is arranged in a hollow channel of the first extension segment and is fixedly connected to a first end of the energy generator; and the second axial fixation member is arranged in a hollow channel of the second extension segment and is fixedly connected to a second end of the energy generator.

As shown in FIG. 7 to FIG. 9, lengths of energy generator and the lens segment are equal in the axial direction, wherein the converging structure of the lens segment is configured to converge energy generated by the energy generator to the target convergence region.

By implementing a periodic functional distribution of axial binary thickness in the balloon, the embodiments of the present disclosure successfully develop the Bessel and Fresnel lens configurations. The balloon provided by the embodiments of the present disclosure is provided with the optical structural characteristics of the Bessel lens and Fresnel lens, which can effectively concentrate the acoustic energy on an appropriate focal depth. The balloon provided by the embodiments of the present disclosure is applied to the ablation surgery. At the ablation radius, the sound pressure significantly increased, and the temperature rise duration is effectively shortened. As a result, the duration of the ablation surgery is significantly reduced, and the surgical effect is significantly improved.

Referring to FIG. 21, the embodiment provides a tubular ultrasonic weak-focusing device, including a balloon 1, wherein the balloon 1 is provided with a transducer 2; the transducer 2 is connected to an inner tube 3; a balloon cavity is arranged between the transducer 2 and the inner wall of the balloon 1; and a coolant can be delivered to the balloon cavity through the inner tube 3.

An acoustic beam adjustment structure is distributed along an axial direction of the balloon 1. The acoustic beam passes through the balloon 1 and the acoustic beam adjustment structure to form an adjustable weak-focusing region on an outer side of the balloon 1, so that the ablation depth and accuracy can be adjusted and the synchronous temperature rise within the target area can be realized.

In an embodiment, the acoustic beam adjustment structure includes at least one or more acoustic unit structures, and the at least one or more acoustic unit structures are located on a balloon wall of the balloon 1.

In an embodiment, the acoustic beam adjustment structure includes at least one or more acoustic unit structures, and the at least one or more acoustic unit structures are located on an outer wall of the transducer 2.

In an embodiment, the acoustic beam adjustment structure includes at least one or more acoustic unit structures, and the at least one or more acoustic unit structures are located in a region between the transducer 2 and the balloon 1.

It will be understood that the acoustic unit structure enters the blood vessel simultaneously with the balloon 1, and will not have a relative displacement with the transducer 2 or the balloon 1, so that the acoustic beam can pass through the acoustic unit structure and the balloon 1 to form an adjustable weak-focusing region at the outer side of the balloon 1.

Further, at least multiple groups of acoustic unit structures are symmetrically arranged along the axial direction of the balloon 1, and the at least multiple groups of acoustic unit structures are located at two ends of the balloon 1.

Further, the acoustic unit structure includes a first medium 41 and a second medium 42, wherein the first medium 41 and the second medium 42 are arranged at intervals; the first medium 41 and the second medium 42 are similar in size; the first medium 41 is in an annular shape and arranged axially along an outer wall of the balloon 1; and the first medium 41 and the balloon 1 are made of the same material.

Increasing the ultrasonic frequency of the transducer 2 results in a larger ablation range or depth. The distances between multiple groups of acoustic unit structures and the length or width of each acoustic unit structure can also be adjusted, thereby adjusting the focusing depth and width of the weak-focusing region. Through forming the weak-focusing region, the ablation depth and accuracy can be adjusted, and the synchronous temperature rise within the target area can be realized. Moreover, it has advantages of uniform and consistent ablation effect and a distinct distal boundary. It can achieve precise ablation for the renal artery branches and the main trunk, which ensures thorough ablation and minimizes damage to the surrounding normal tissues as much as possible. It meets the clinical requirement of “ablation as complete as possible”, thereby improving the effectiveness and safety of the treatment.

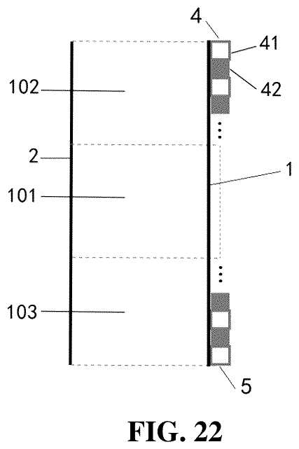

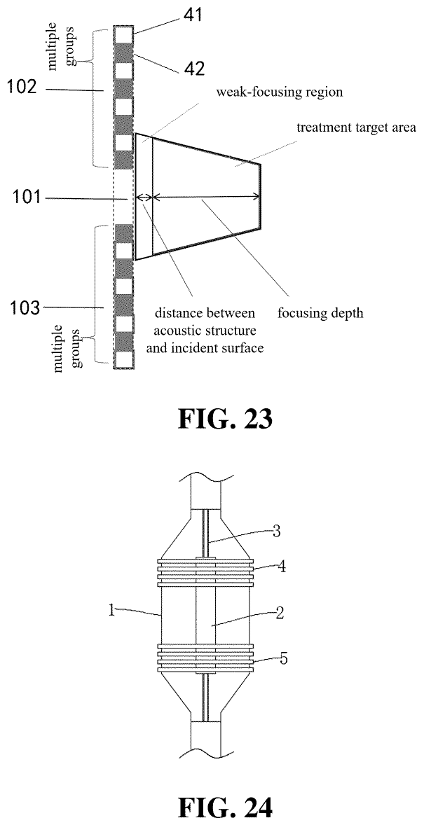

In a specific embodiment, referring to FIG. 22-FIG. 24, the acoustic beam adjustment structure includes a first acoustic unit structure 4 and a second acoustic unit structure 5, wherein the first acoustic unit structure 4 and the second acoustic unit structure 5 are respectively located on two ends of the balloon 1; the first acoustic unit structure 4 and the second acoustic unit structure 5 are arranged symmetrically along the axial direction of the balloon 1; and the first acoustic unit structure 4 and the second acoustic unit structure 5 have a gap therebetween.

In the embodiment, a first coherence zone 102 is formed by the balloon cavity between the first acoustic unit structure 4 and the transducer 2, and the width of the first coherence zone 102 is approximately equal to the length of the first acoustic unit structure 4. When the transducer 2 operates, a first acoustic beam is formed in the first coherence zone 102, and the first acoustic beam propagates along the radial direction of the balloon 1.

After the first acoustic beam passes through the balloon 1 and the first acoustic unit structure 4, it is refracted by the first acoustic unit structure 4 to form two acoustic beams, including a fourth acoustic beam and a fifth acoustic beam.

The fourth acoustic beam propagates along a direction forming an obtuse angle with the radial angle of the balloon 1, which can be understood as the fourth acoustic beam being propagated in a direction away from the radial center axis of the balloon 1.

The fifth acoustic beam propagates along a direction forming an acute angle with the radial angle of the balloon 1, which can be understood as the fifth acoustic beam being propagated in a direction close to the radial center axis of the balloon 1.

In the embodiment, a second coherence zone 103 is formed by the balloon cavity between the second acoustic unit structure 5 and the transducer 2, and the width of the second coherence zone 103 is approximately equal to the length of the second acoustic unit structure 5. When the transducer 2 operates, a third acoustic beam is formed in the second coherence zone 103, and the third acoustic beam propagates along the radial direction of the balloon 1.

After the second acoustic beam passes through the balloon 1 and the second acoustic unit structure 5, it is refracted by the second acoustic unit structure 5 to form another two acoustic beams, including a sixth acoustic beam and a seventh acoustic beam.

The target propagates along a direction forming an obtuse angle with the radial angle of the balloon 1, which can be understood as the seventh acoustic beam being propagated in a direction away from the radial center axis of the balloon 1.

The sixth acoustic beam propagates along a direction forming an acute angle with the radial angle of the balloon 1, which can be understood as the sixth acoustic beam being propagated in a direction close to the radial center axis of the balloon 1.

In the embodiment, the balloon cavity at the spacing between the first acoustic unit structure 4 and the second acoustic unit structure 5 forms a free propagation zone 101, and the width of the free propagation zone 101 is approximately equal to the length of the spacing between the first acoustic unit structure 4 and the second acoustic unit structure 5 When the transducer 2 operates, the second acoustic beam is formed win the free propagation zone 101, and the second acoustic beam propagates along the radial direction of the balloon 1.

After the second acoustic beam passes through the balloon 1, since the medium outside the balloon 1 at the free propagation zone 101 is human tissue on the vascular wall, the second acoustic beam will not have significant refraction. The second acoustic beam, along with the fifth acoustic beam and the sixth acoustic beam, forms a weak-focusing region with a larger incident surface width near the balloon 1 and a smaller incident surface width away from the balloon 1. It is understandable that the longitudinal axis of the weak-focusing region forms a shape that is similar to a trapezoidal structure. Furthermore, it can be known that due to the symmetrical arrangement of the first acoustic unit structure 4 and the second acoustic unit structure 5, the formed trapezoidal structure is similar to an isosceles trapezoidal structure, which is used to ablate the 103.

Currently, in order to increase the range or depth of ablation, the technical means adopted are to increase the ultrasonic frequency of transducer 2. The greater the ultrasonic frequency is, the greater the range or depth of ablation is, but the disadvantage is that the mechanical wave vibration causes greater tissue heating; and at the same time, the accuracy of ablation will also be decreased.

In this embodiment, the positions of the first acoustic unit structure 4 and the second acoustic unit structure 5 in the axial direction of the balloon 1 are adjustable. By adjusting the distance between the first acoustic unit structure 4 and the second acoustic unit structure 5, the width of the weak-focusing region close to the balloon 1 and the width away from the balloon 1 can be adjusted.

When the distance between the first acoustic unit structure 4 and the second acoustic unit structure 5 increases, the incident surface width of the weak-focusing region near the balloon 1 becomes larger, and the incident surface width of the weak-focusing region away from the balloon 1 also increases. This results in a larger ablation area, accompanied by a relative reduction in ablation precision.

When the distance between the first acoustic unit structure 4 and the second acoustic unit structure 5 decreases, the incident surface width of the weak-focusing region near the balloon 1 becomes smaller, and the incident surface width of the weak-focusing region away from the balloon 1 also decreases. This results in a smaller ablation area, accompanied by relative increasing in the ablation precision.

It can further be known that the heights of the first acoustic unit structure 4 and the second acoustic unit structure 5 in the axial direction of the balloon 1 are adjustable. By adjusting the heights of the first acoustic unit structure 4 and the second acoustic unit structure 5, the incident surface width of the weak-focusing region close to the balloon 1 and the incident surface width away from the balloon 1 can be adjusted.

When the heights of the first acoustic unit structure 4 and the second acoustic unit structure 5 increase, the incident surface width of the weak-focusing region near the balloon 1 becomes larger, and the incident surface width of the weak-focusing region away from the balloon 1 also increases. This results in a larger ablation area, accompanied by a relative reduction in ablation precision.

When the heights of the first acoustic unit structure 4 and the second acoustic unit structure 5 decrease, the incident surface width of the weak-focusing region near the balloon 1 becomes smaller, and the incident surface width of the weak-focusing region away from the balloon 1 also decreases. This results in a smaller ablation area, accompanied by relative increasing in the ablation precision.

It is further known that the widths of the first acoustic unit structure 4 and the second acoustic unit structure 5 in the horizontal direction are both adjustable When the first acoustic unit structure 4 and the second acoustic unit structure 5 are fixed at the axial position of the balloon 1, and the formed depth of the weak-focusing region is constant, by adjusting the widths of the first acoustic unit structure 4 and the second acoustic unit structure 5, the distance between the first acoustic unit structure 4 and the second acoustic unit structure 5 and the incident surface of the treatment area can be adjusted, thereby adjusting the focusing depth.

By adjusting the ultrasonic frequency, the distance between the acoustic structure and the incident surface of the treatment area, the number of the first acoustic unit structure 4 and the second acoustic unit structure 5, and the heights or widths of the first acoustic unit structure 4 and the second acoustic unit structure 5, the focusing depth can be varied in a range of 3 to 10 mm. The focusing depth is defined as the radial distal end at the axial symmetric center where the field strength is half of the peak field strength in the treatment area.

In the embodiment, the weak-focusing region is formed by the first acoustic unit structure 4 and the second acoustic unit structure 5, so that the ablation depth and accuracy can be adjusted and the synchronous temperature rise within the target area can be realized. Moreover, it has advantages of uniform and consistent ablation effect and a distinct distal boundary. It can achieve precise ablation for the renal artery branches and the main trunk, which ensures thorough ablation and minimizes damage to the surrounding normal tissues as much as possible. It meets the clinical requirement of “ablation as complete as possible”, thereby improving the effectiveness and safety of the treatment.

It is understandable that in other embodiments, in the specific implementation, the first acoustic unit structure 4 and the second acoustic unit structure 5 can also be asymmetrically arranged. The heights and widths of the first acoustic unit structure 4 and the second acoustic unit structure 5 can be different, and the longitudinal axis of the formed weak-focusing region is also a trapezoidal structure, which is used to ablate the treatment target area.

In the embodiment, the transducer 2 is a tubular transducer, and the transducer 2 is sleeved on the inner tube 3 in the axial direction, which has a simple structure, low cost, and stable operation performance.

Furthermore, the second acoustic unit structure 5 is symmetrically arranged with the first acoustic unit structure 4, and the first acoustic unit structure 4 includes the first medium 41 and the second medium 42. One first medium 41 and one second medium 42 form the smallest unit, and the first medium 41 and the second medium 42 are similar in size. Several smallest units are arrayed to form the first acoustic unit structure 4. The material sound velocity of the first medium 41 is C1, and the material sound velocity of the second medium 42 is C2. The frequency of the acoustic wave emitted by the transducer 2 is f, and the preset height of the minimal unit composed of the first medium 41 and the second medium 42 is d, where

$$d = \frac{C 1 * C 2}{2 ( C 1 - C 2 ) f} .$$

When the wave beam propagates in the smallest unit at a height of d, the split wave beams passing through the first medium 41 and the second medium 42 will generate a phase difference ranging from approximately π/2 to 3π/2. Preferably, the split beams of the first medium 41 and the second medium 42 generate a phase difference close to, and the width w of the minimal unit is equal to or close to d.

In the embodiment, the first medium 41 is of the same material as the balloon 1, and the first medium 41 is arranged in an annular shape on the outer wall of the balloon 1. Multiple annular first mediums 41 are arranged along the axial direction on the outer wall of the balloon 1, and there is a gap between two adjacent first mediums 41.

In an embodiment, the first medium 41 and the balloon 1 are both made of nylon, and the first medium 41 and the balloon 1 are integrally molded, which can save processing costs.

In the embodiment, the second medium 42 is the human tissue on the vascular wall. When the balloon 1 enters the blood vessel, the human tissue of the vascular wall is embedded in the gap between the first medium 41, thereby forming the second medium 42.

During the specific implementation of the treatment, the transducer 2 generates acoustic beams. The first acoustic beam passes through the first medium 41 and the second medium 42 of the first acoustic unit structure 4 and is refracted into two beams, including a fourth acoustic beam and a fifth acoustic beam.

The fourth acoustic beam propagates along a direction forming an obtuse angle with the radial angle of the balloon 1, which can be understood as the fourth acoustic beam being propagated in a direction away from the radial center axis of the balloon 1.

The fifth acoustic beam propagates along a direction forming an acute angle with the radial angle of the balloon 1, which can be understood as the fifth acoustic beam being propagated in a direction close to the radial center axis of the balloon 1.

After the second acoustic beam passes through the balloon 1 and the second acoustic unit structure 5, it is refracted by the second acoustic unit structure 5 to form another two acoustic beams, including a sixth acoustic beam and a seventh acoustic beam.

The seventh acoustic beam propagates along a direction forming an obtuse angle with the radial angle of the balloon 1, which can be understood as the seventh acoustic beam being propagated in a direction away from the radial center axis of the balloon 1.

The sixth acoustic beam propagates along a direction forming an acute angle with the radial angle of the balloon 1, which can be understood as the sixth acoustic beam being propagated in a direction close to the radial center axis of the balloon 1.

After the second acoustic beam passes through the balloon 1, since the medium outside the balloon 1 at the free propagation zone 101 is human tissue on the vascular wall, the second acoustic beam will not have significant refraction. The second acoustic beam, along with the fifth acoustic beam and the sixth acoustic beam, forms a weak-focusing region with a larger incident surface width near the balloon 1 and a smaller incident surface width away from the balloon 1. It is understandable that the longitudinal axis of the weak-focusing region forms a shape that is similar to a trapezoidal structure. Furthermore, it can be known that due to the symmetrical arrangement of the first acoustic unit structure 4 and the second acoustic unit structure 5, the formed trapezoidal structure is similar to an isosceles trapezoidal structure, which is used to ablate the treatment target area.

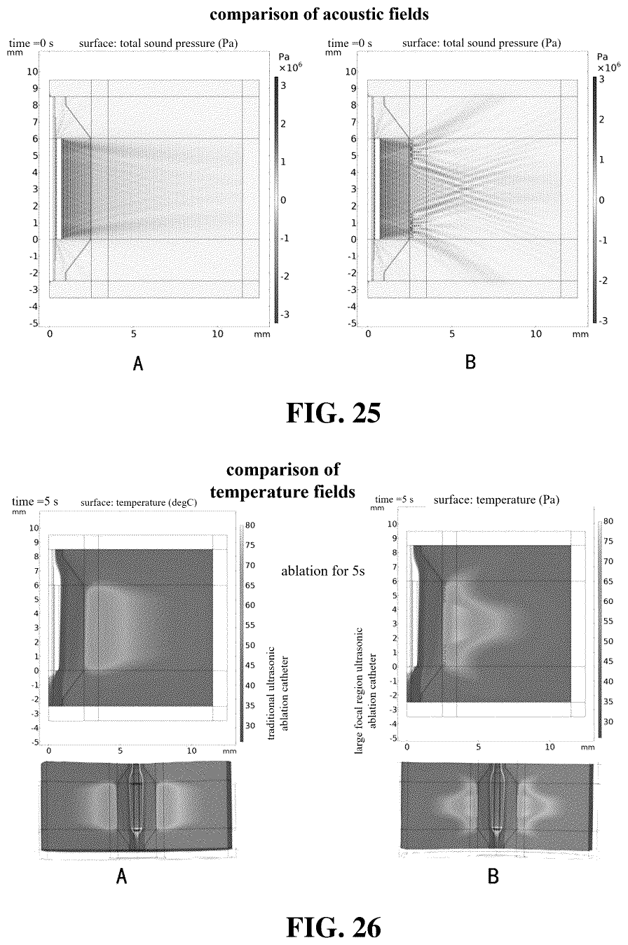

Referring to FIG. 25, FIG. A shows that in the prior art, the balloon is not provided with an acoustic beam adjustment structure, and the sound field generated by the transducer is relatively uniform.

FIG. B shows that in the present disclosure, the balloon is provided with an acoustic beam adjustment structure, and the sound field generated by the transducer has a very distinct weak-focusing region.

The present disclosure further provides a tubular ultrasonic weak-focusing method which uses the above tubular ultrasonic weak-focusing device to ablate the treatment target area.

Referring to FIG. 26, FIG. A shows an uniform distribution of temperature field during the 5—second ablation process of the traditional ultrasonic ablation catheter in the prior art.

FIG. B shows the balloon provided with an acoustic beam adjustment structure in the present disclosure, resulting in a distinct weak-focusing region in the temperature field.

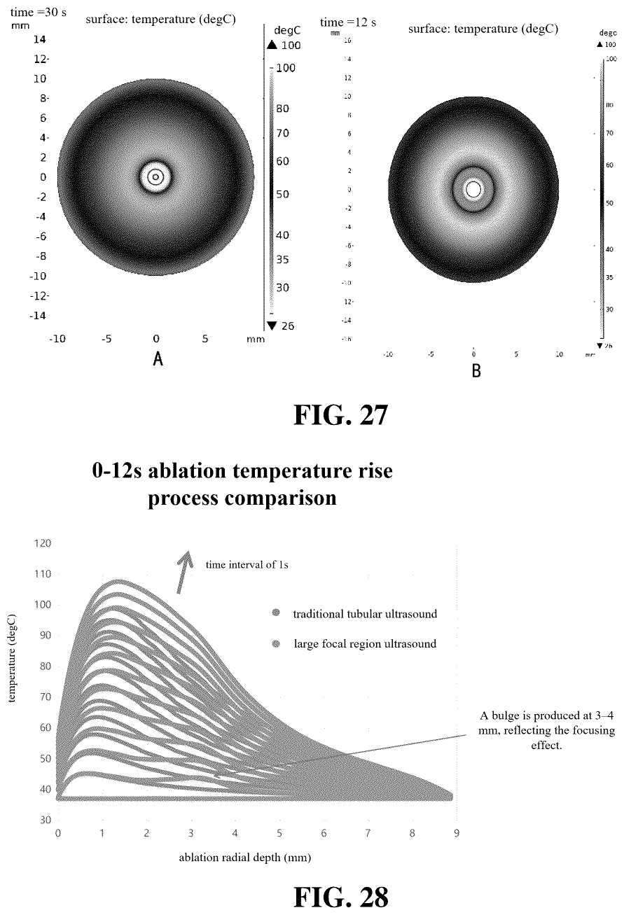

Referring to FIG. 27-FIG. 28, in FIG. A, the conventional ultrasonic ablation catheter in the prior art shows that the temperature rise in the treatment area expands from the inside outward, and the duration of the temperature rise is relatively long.

FIG. B shows that in the present disclosure, the balloon is provided with an acoustic beam adjustment structure. The ultrasonic ablation catheter simultaneously heats up in the treatment area, and the temperature rise duration is shorter. The ablation radial depth is greater, and the effect is better. A bulge is produced at 3-4 mm, which reflects the focusing effect.

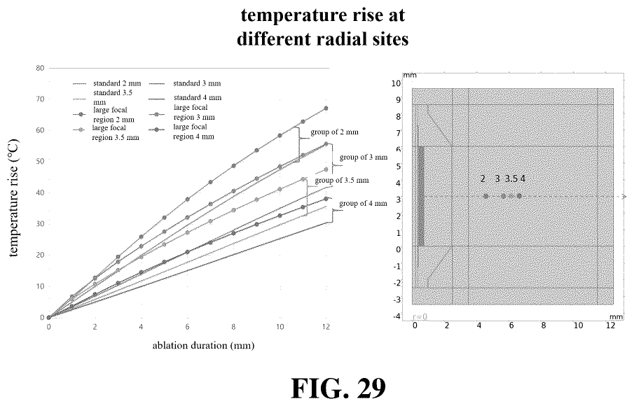

Referring to FIG. 29, the ultrasonic ablation catheter of the present disclosure exhibits a temperature rise rate approximately 1.5-2 times that of the traditional ultrasonic ablation catheter during the initial stage (within 4 seconds), thereby achieving faster temperature rise.

Understandably, the tubular ultrasonic weak-focusing device of the present disclosure can also be applied to renal artery denervation for hypertension, pulmonary artery denervation for pulmonary hypertension, hepatic artery denervation for diabetes mellitus, visceral microneural ablation for heart failure, and targeted pulmonary denervation for chronic obstructive pulmonary disease.

In the text, relationship terms such as first and second, etc., are used merely to distinguish one entity or operation from another, and do not necessarily require or imply the existence of any this actual relationship or order between those entities or operations. Furthermore, the terms “include”, “comprise” or any other variations thereof are intended to cover a non-exclusive inclusion, so that a process, method, object, or device including a series of elements includes not only those elements, but also other elements that are not explicitly listed, or the components that are inherent to this process, method, object, or device. Without further limitation, the component defined by a phrase “including . . . ” does not exclude that the other same elements also exist in the process, method, object, or device including the component.

The foregoing is merely preferable embodiments of the present disclosure, and is not intended to limit the scope of the protection of the present disclosure. For those skilled in the art, the present disclosure may have various changes and variations. Any modifications, equivalent substitutions, improvements, etc., made within the spirit and principles of the present disclosure, shall be included in the scope of protection of the present disclosure.

1. A balloon, wherein the balloon in an expanded state comprises: a first extension segment, a lens segment, and a second extension segment;
the lens segment is arranged between the first extension segment and the second extension segment in an axial direction; and
the lens segment comprises a converging structure prepared from an energy-permeable material, wherein
the converging structure is configured to converge energy from a hollow channel of the lens segment to a target convergence region.

2. The balloon according to claim 1, wherein the converging structure comprises a plurality of first thickness segments and a plurality of second thickness segments;
radial dimensions of the first thickness segments and the second thickness segments are different; and
the plurality of first thickness segments and the plurality of second thickness segments are arranged alternately in the axial direction.

3. The balloon according to claim 1, wherein the lens segment comprises a middle length segment; and
the middle length segment is arranged in an axial middle part of the lens segment, and the converging structure is arranged on two sides of the middle length segment.

4. The balloon according to claim 1, wherein the balloon is a non-compliant balloon.

5. A balloon catheter, wherein the balloon catheter comprises a balloon, an energy generator, a first axial fixation member, and a second axial fixation member, and the balloon in an expanded state comprises: a first extension segment, a lens segment, and a second extension segment, wherein the lens segment is arranged between the first extension segment and the second extension segment in an axial direction; and
the energy generator is arranged in a hollow channel of the lens segment, and the energy generator has a length in the axial direction corresponding to a length of the lens segment;
the first axial fixation member is arranged in a hollow channel of the first extension segment and is fixedly connected to a first end of the energy generator; the second axial fixation member is arranged in a hollow channel of the second extension segment and is fixedly connected to a second end of the energy generator, wherein
the converging structure of the lens segment is configured to converge energy generated by the energy generator to a target convergence region.

6. The balloon catheter according to claim 5, wherein the converging structure comprises first thickness segments with a base thickness d1 and second thickness segments with a thickness d2, wherein
the thickness d2 of the second thickness segments is obtained by adding a phase difference thickness d to the base thickness d1, and the phase difference thickness d is determined based on a phase difference between adjacent wave groups generated by the energy generator.

7. The balloon catheter according to claim 6, wherein lengths of the first thickness segments and the second thickness segments in the axial direction are approximate or equal to the phase difference thickness d.

8. The balloon catheter according to claim 7, wherein focusing characteristics presented by the converging structure are consistent with focusing characteristics of the quasi-Bessel lens; and
a middle length segment of the lens segment is determined based on a position of the target convergence region.

9. The balloon catheter according to claim 7, wherein the focusing characteristics presented by the converging structure are consistent with focusing characteristics of the quasi-Bessel lens; and
the number of the first thickness segments and the second thickness segments are n and n+1 respectively, wherein n is determined based on a position of the target convergence region.

10. The balloon catheter according to claim 9, wherein the converging structure comprises n first thickness segments and n+1 second thickness segments, wherein
when a distance between a center point at the position of the target convergence region in a radial direction and an outer surface of the lens segment is in a range of [5.7 mm, 6.3 mm], n is set to 11.

11. The balloon catheter according to claim 6, wherein focusing characteristics presented by the converging structure are consistent with focusing characteristics of the Fresnel lens; and
lengths of the first thickness segments, the second thickness segment, and a middle length segment in the axial direction are determined based on a position of the target convergence region according to a Fresnel zone plate formula.

12. The balloon catheter according to claim 8, wherein the position of the target convergence region is determined based on a target physiological position to be ablated.

13. The balloon catheter according to claim 12, wherein the target physiological position comprises sympathetic nerve positions or parasympathetic nerve positions of a renal artery, duodenum, celiac artery, abdominal aorta, common hepatic artery, and hepatic innominate artery.

14. A tubular ultrasonic weak-focusing device, comprising a balloon, wherein a transducer is arranged in the balloon; the transducer emits an acoustic beam for ablation; an acoustic beam adjustment structure is distributed along an axial direction of the balloon; and
the acoustic beam passes through the balloon and the acoustic beam adjustment structure to form an adjustable weak-focusing region on an outer side of the balloon.

15. The tubular ultrasonic weak-focusing device according to claim 14, wherein the acoustic beam adjustment structure comprises at least one or more acoustic unit structures, and the at least one or more acoustic unit structures are located on a balloon wall of the balloon.

16. The tubular ultrasonic weak-focusing device according to claim 14, wherein the acoustic beam adjustment structure comprises at least one or more acoustic unit structures, and the at least one or more acoustic unit structures are located on an outer wall of the transducer.

17. The tubular ultrasonic weak-focusing device according to claim 14, wherein the acoustic beam adjustment structure comprises at least one or more acoustic unit structures, and the at least one or more acoustic unit structures are located in a region between the transducer and the balloon.

18. The tubular ultrasonic weak-focusing device according to claim 14, wherein at least multiple groups of acoustic unit structures are symmetrically arranged along the axial direction of the balloon, and the at least multiple groups of acoustic unit structures are located at two ends of the balloon.

19. The tubular ultrasonic weak-focusing device according to claim 14, wherein the acoustic unit structure comprises a first medium and a second medium, and the first medium and the second medium are arranged at intervals.

20. A tubular ultrasonic weak-focusing method, using the tubular ultrasonic weak-focusing device according to claim 14 to ablate a treatment target area.
